(19) United States
(12) Patent Application Publication
(10) Pub. No.: US 2012/0157667 A1
(43) Pub. Date: Jun. 21, 2012

(54) Methods Of Producing Humanized Non-Human Mammals

(76) Inventors: Qingfeng Chen, Singapore (SG); Jianzhu Chen, Cambridge, MA (US)

(21) Appl. No.: 13/381,313

(22) PCT Filed: Jun. 28, 2010
(86) PCT No.: PCT/US2010/040260

§ 371 Date: Mar. 12, 2012

Related U.S. Application Data
(60) Provisional application No. 61/221,438, filed on Jun. 29, 2009.

(51) Int. Cl. C07K 16/24; C12P 21/08; C12N 5/18; A01K 67/027

(52) U.S. Cl. 530/388.23; 800/21; 800/6; 435/335

(57) ABSTRACT

Provided herein are methods of reconstituting functional human blood cell lineages in a non-human mammal comprising introducing human hematopoietic stem cells (HSCs) and nucleic acid encoding one or more human cytokines into an immunodeficient non-human mammal. The non-human mammal is maintained under conditions in which the nucleic acid is expressed and the human HSCs differentiate into functional human blood cell lineages in the non-human mammal, thereby reconstituting functional human blood cell lineages in the non-human mammal. Also provided are methods of producing human antibodies directed against an immunogen in a non-human mammal, hybridomas that secrete the monoclonal antibodies as well as antibodies (e.g., polyclonal antibodies; monoclonal antibodies) produced by the B cells and non-human mammals produced by the methods.

### RELATED APPLICATION(S)

This application claims the benefit of U.S. Provisional Application No. 61/221,438, filed on Jun. 29, 2009. The entire teachings of the above application(s) are incorporated herein by reference.

### BACKGROUND OF THE INVENTION

There is a great need to study the human immune response to pathogen infections in a small animal model in a systematic and controlled manner. Over the past two decades, tremendous efforts have been devoted to reconstitute severe combined immunodeficient (scid) mice, which lack T and B lymphocytes, with human-blood lineage cells (Shultz L D, Ishikawa F, Greiner D L (2007) Nat Rev Immunol 7:118-130). However, early attempts were unsuccessful because of poor engraftment, rapid disappearance of human T and B cells, or rapid development of hematopoietic malignancies in the recipient mice. A breakthrough was achieved by using recipient mice that are deficient not only in T and B because of either the scid mutation or mutation of the recombination activating gene (Rag), but also in natural killer (NK) cells because of the deletion of the common gamma chain (γc or Il2rg) (Hiramatsu H, et al. (2003) Blood 102:873-880; Traggiai E, et al. (2004) Science 304:104-107). Adoptive transfer of human hematopoietic stem cells (HSCs) into either NOD-scid Il2rg^{-/-} (NSG) recipients or BALB/c-Rag2^{-/-} Il2rg^{-/-} recipients leads to stable, long-term engraftment of HSCs in the recipient bone marrow (BM) and generation of all human-blood lineage cells in the periphery (humanized mice or humice) (Hiramatsu H, et al. (2003) Blood 102:873-880; Traggiai E, et al. (2004) Science 304:104-107).

The existing humanized mouse models provide an important tool to study infection by human pathogens (Davis P H, Stanley S L, Jr. (2003) Cell Microbial 5:849-860; Bente D A, et al. (2005) J Virol 79:13797-13799; Islas-Ohlmayer M, et al. (2004) J Virol 78:13891-13900; Guirado E, et al. (2006) Microbes Infect 8:1252-1259; Kneteman N M, et al. (2006) Hepatology 43:1346-1353; Jiang Q, et al. (2008) Blood 112:2858-2868), especially those that infect human-blood lineage cells. They also begin to allow investigations of the human immune response to pathogens in a small animal model. However, the currently available models are far from optimal. For example, the level of human cell reconstitution differs markedly among different cell lineages. The reconstitution of B cells is robust and the reconstitution of T cells is reasonable, but the B cells and T cells are not functional. In addition, the reconstitution of NK cells and myeloid lineage cells is generally poor or undetectable.

Thus, there is a great need for improved non-human models of the human immune system and methods of producing them.

### SUMMARY OF THE INVENTION

Shown herein is that the poor reconstitution of human blood cell lineages by human hematopoietic stem cells (HSCs) is mainly the result of a deficiency of the appropriate human cytokines that are necessary for the development and maintenance of these cell lineages in the non-human mammal. When plasmid DNA encoding human IL-15 and Flt-3/Flk-2 ligand were delivered into humanized mice (e.g., by hydrodynamic tail-vein injection), the expression of the human cytokines lasted for 2 to 3 weeks, and elevated levels of NK cells were induced for more than a month. The cytokine-induced NK cells expressed both activation and inhibitory receptors, killed target cells in vitro, and responded robustly to a virus infection in vivo. Similarly, expression of human GM-CSF and IL-4, macrophage colony stimulating factor, or erythropoietin and IL-3 resulted in significantly enhanced reconstitution of dendritic cells, monocytes/macrophages, or erythrocytes, respectively (see Chen, Q., et al., Proc. Natl. Acad. Sci., USA, 106:21783-21788 (2009) which is incorporated herein by reference). Also, GM-CSF and IL-4 enhanced human T cell and human B cell reconstitution. Thus, using human cytokine gene expression (e.g., by hydrodynamic delivery) along with human HSCs is a simple and efficient method to improve reconstitution of specific human-blood cell lineages in humanized mice, providing an important tool for modeling human diseases and their progression and studying human immune responses in a small animal model.

Accordingly, provided herein are methods of reconstituting functional human blood cell lineages in a non-human mammal, thereby producing a humanized non-human mammal. In particular embodiments, humanized mice (humice) are produced.

In one aspect, the invention is directed to a method of reconstituting functional human blood cell lineages in a non-human mammal comprising introducing human hematopoietic stem cells (HSCs) and a (one or more) nucleic acid encoding one or more human cytokines into an immunodeficient non-human mammal. The non-human mammal is maintained under conditions in which the nucleic acid is expressed, and the human HSCs differentiate into functional human blood cell lineages in the non-human mammal, thereby reconstituting functional human blood cell lineages in the non-human mammal.

In another aspect, the invention is directed to a method of reconstituting functional human NK cells in a non-human mammal comprising introducing into an immunodeficient non-human mammal human hematopoietic stem cells (HSCs) and nucleic acid encoding one or more human cytokines, wherein the human cytokines promote differentiation of the human HSCs into functional human NK cells when expressed in the non-human mammal. The non-human mammal is maintained under conditions in which the nucleic acid is expressed and the human HSCs differentiate into functional human NK cells in the non-human mammal, thereby enhancing reconstitution of human NK cells in the non-human mammal.

In yet another aspect, the invention is directed to a method of reconstituting functional human dendritic cells in a non-human mammal comprising introducing into an immunodefficient non-human mammal human hematopoietic stem cells (HSCs) and nucleic acid encoding one or more human cytokines, wherein the human cytokines promote differentiation of the human HSCs into functional human dendritic cells when expressed in the non-human mammal. The non-human mammal is maintained under conditions in which the nucleic acid is expressed, and the human HSCs differentiate into functional human dendritic cells in the non-human mammal, thereby enhancing reconstitution of functional human dendritic cells in the non-human mammal.

In another aspect, the invention is directed to a method of reconstituting functional human monocytes/macrophages in a non-human mammal comprising introducing into an immunodeficient non-human mammal human HSCs and nucleic acid encoding one or more human cytokines, wherein the human cytokines promote differentiation of the human HSCs into functional human monocytes/macrophages when expressed in the non-human mammal. The non-human mammal is maintained under conditions in which the nucleic acid is expressed and the human HSCs differentiate into functional human monocytes/macrophages in the non-human mammal, thereby reconstituting functional human monocytes/macrophages in the non-human mammal.

In another aspect, the invention is directed to a method of reconstituting functional human erythrocytes in a non-human mammal comprising introducing into an immunodeficient non-human mammal human HSCs and nucleic acid encoding one or more human cytokines, wherein the human cytokines promote differentiation of the human HSCs into functional human erythrocytes when expressed in the non-human mammal. The non-human mammal is maintained under conditions in which the nucleic acid is expressed and the human HSCs differentiate into functional human erythrocytes in the non-human mammal, thereby reconstituting functional human erythrocytes in the non-human mammal.

In another aspect the invention is directed to a method of reconstituting functional human T cells and human B cells in a non-human mammal comprising introducing human hematopoietic stem cells (HSCs) and nucleic acid encoding one or more human cytokines into an immunodeficient non-human mammal, wherein the human cytokines promote differentiation of the human HSCs into functional human T cells and human B cells when expressed in the non-human mammal. The non-human mammal is maintained under conditions in which the nucleic acid is expressed and the human HSCs differentiate into functional human T cells and human B cells in the non-human mammal, thereby reconstituting functional human T cells and human B cells in the non-human mammal.

In another aspect, the invention is directed to a method of producing human antibodies directed against an immunogen in a non-human mammal comprising introducing human hematopoietic stem cells (HSCs) and nucleic acid encoding one or more human cytokines into an immunodeficient non-human mammal wherein the human cytokines promote differentiation of the human HSCs into functional human T cells and functional human B cells. The non-human mammal is maintained under conditions in which the nucleic acid is expressed and the human HSCs differentiate into functional human T cells and functional human B cells in the non-human mammal. The non-human mammal is immunized with the immunogen and maintained under conditions in which the human B cells produce human antibodies directed against the immunogen in the non-human mammal, thereby producing human antibodies directed against the immunogen in the non-human mammal. B cells that produce antibody directed against the immunogen can be further isolated and used to produce hybridomas that secrete monoclonal antibodies directed against the immunogen.

Hybridomas that secrete the monoclonal antibodies as well as antibodies (e.g., polyclonal antibodies; monoclonal antibodies) produced by the B cells are also encompassed by the invention. The non-human mammals produced by the methods provided herein are also encompassed by the invention.

The methods described herein provide a simple and efficient methods to reconstitute functional human blood cell lineages (e.g., myeloid cells; lymphoid cells) in a non-human mammal (e.g., a humanized mouse) and to produce human antibodies in non-human mammals.

### BRIEF DESCRIPTION OF THE DRAWINGS

FIGS. 1A-1B: Human CD34+ cells from the bone marrow (BM) of humice can be stimulated to differentiate into NK cell in vitro. (FIG. 1A) Comparison of CD34 versus CD133 staining profiles of mononuclear cells from the BM of humice (Left) and after purification with anti-CD34 beads by MACS (Right). Events shown are pre-gated on human CD45⁺ cells. The numbers indicate percentages of cells in the gated region. Representative data from one of four mice are shown. (FIG. 1B) NKp46 versus CD56 staining profiles of human CD34⁺ cells cultured in the absence (Ctrl) or presence of IL-15 and FL for 7 days. The numbers indicate the percentages of CD56⁺ NKp46⁺ cells.

FIG. 2: Hydrodynamic injection-mediated gene delivery creates a systemic human cytokine environment in mouse. pcDNA vectors expressing human IL-15 and FL were mixed together (each 50 μg) and injected into humice hydrodynamically. The levels of human IL-15 and FL in mouse sera were analyzed by ELISA at the indicated time points (n=3 for each timepoint).

FIGS. 3A-3C: Expression of IL-15 and FL stimulates human NK cell development in vivo. The empty pcDNA vector (Ctrl) or pcDNA vectors expressing IL-15 and/or FL were hydrodynamically injected into humanized mice. Nine days later, cells were prepared from the indicated tissues and stained for human CD45, CD3, and CD56. (FIG. 3A) Dot plots show CD3 versus CD56 staining profiles gating on CD45⁺ cells. The numbers indicate percentages of CD56⁺CD3⁻ cells in the gated region. Representative data from one of five mice per group are shown. Humice were constructed with three different donor HSCs. (FIG. 3B) Comparison of frequencies (mean±SEM) of CD56⁺CD3⁻ NK cells within CD45⁺ human leukocytes in various organs 9 days after cytokine gene delivery (n=5), (FIG. 3C) The frequencies (mean±SEM) of CD56⁺CD3⁻ NK cells within CD45⁺ human leukocytes in the blood overtime (n=3).

FIGS. 4A-4E: Human NK cells are functional. (FIG. 4A) NK cells mediate liver damage following adenovirus infection in humice. Nine days after cytokine gene delivery, PBS or replication-deficient adenovirus (Ad) were hydrodynamically injected into the humice. Livers of humice were collected for H&E staining 3 days after infection (n=3 for each group). The arrows indicate areas of necrosis and leukocyte infiltration. Ctrl, humice without cytokine gene delivery; IL-15/FL, humice with cytokine gene delivery. Magnifications are shown. (FIG. 4B) Comparison of ALT levels in the serum. Sera were collected from adenovirus or PBS-treated humice 5 days after infection and assayed for ALT activity (mean±SEM, n=3 per group). P<0.05 between IL-15/FL/Ad and other groups. (FIG. 4C) Serum levels of IFN-γ in adenovirus infected humice. Sera were collected from adenovirus-infected humice and measured for IFN-γ by ELISA. Mean±SEM is shown (n=3 per group). P<0.05. (FIG. 4D) The number of MNCs in the livers of various humice. Livers were harvested 5 days after adenovirus infection. Total hepatic MNCs were counted and the number of human CD45⁺ cells was determined by flow cytometry analysis (mean±SEM, n=3 per group). P<0.05 between IL-15/FL/Ad and other groups. (FIG. 4E) Localization of CD56⁺ human NK cells to the lesions in the liver. Liver tissues were embedded in paraffin, sectioned, stained for CD56, and analyzed by microscopy. Arrows indicate the regions of lesion. Representative images are shown from one of three mice.

FIGS. 5A-5C: Induction of specific human blood lineage cells by corresponding human cytokine gene delivery. (FIG. 5A) Improved reconstitution of dendritic cells. Humice were hydrodynamically injected with empty pcDNA vector or pcDNA vectors expressing the indicated human cytokine genes. Nine days after injection, single-cell suspension was prepared from various organs and stained for human CD45, CD 11c, and CD209. Shown are CD209 versus CD11c staining profiles gating on CD45⁺ human cells. Representative data from one of five mice are shown. (FIG. 5B) Improved reconstitution of monocytes/macrophages. The experiments were carried out the same as in (FIG. 5A), except pcDNA-encoding M-CSF were injected and cells were stained for human CD45 and CD14. Shown are CD14 versus CD45 staining profiles gating on CD45⁺ human cells. Representative data from one of three mice are shown. (FIG. 5C) Improved reconstitution of erythrocytes. The experiments were carried out the same as in FIG. 5A, except pcDNA-encoding EPO and IL-3 were injected and blood was stained for human CD235ab 7 and 30 days after injection. Shown are CD235ab versus DAPI staining profiles of all blood cells. Representative data from one of three mice are shown. The numbers indicate percentages of cells in the gated region.

FIGS. 6A-6B: Reconstitution of human NK cells in humanized mice. (FIG. 6A) Twelve weeks after HSC engraftment, the reconstitution of human cell lineages in mononuclear cells of peripheral blood were analyzed by flow cytometry. Dot plots show staining profiles of human CD45 versus mouse CD45 gating on live nucleated cells, or staining profiles of CD3 versus CD19 or CD14 versus CD56 gating on CD45+ human cells. (FIG. 6B) CD14 versus CD56 staining profiles of human CD45+ cells in the blood, bone marrow, spleen, lung, and liver of a humanized mouse. The numbers indicate percentage of cells in the gated region. Representative data from one of six mice are shown.

FIGS. 7A-7B: Increased IL-15 levels in the circulation when expressed using IL-2 signal peptide sequence. (FIG. 7A) Schematic diagrams of IL-15 expressing vectors. IL-15 gene, with either its endogenous signal sequence (SP) or IL-2SP, was cloned into pcDNA vector with a CMV promoter. (FIG. 7B) Comparison of serum level of IL-15. An empty pcDNA vector (Ctrl), pcDNA vector encoding IL-15, and pcDNA vector encoding IL-15 with an IL-2 signal sequence were hydrodynamically injected into NSG mice. Seven days after injection, sera were collected and assayed for IL-15 level by ELISA.

FIGS. 8A-8G: Increased numbers of human cells following IL-15 and FL expression. (FIGS. 8A-8G) The empty pcDNA vector (Ctrl) or pcDNA vectors expressing both IL-15 and FL were hydrodynamically injected into humanized mice. Nine days later, cells were prepared from the indicated tissues and stained for human CD45 plus CD3, CD56, CD14, CD11c, CD1c, ILT7, CD303, and CD19. Absolute numbers of human CD45+ leukocyte, CD56+ NK cells, CD11c+CD1c+ dendritic cells, ILT7+CD303+ plasmacytoid dendritic cells, CD14+ monocytes/macrophages, CD3+ T cells, and CD19+ B cells in various organs were calculated by multiplying the total cell numbers with the frequency of the specific cell types. Shown are mean±SEM (n=3). Numbers of cells in the bone marrow (BM) were from two femurs.

FIG. 9: Cell surface phenotype of human NK cells in IL-15 and FL treated humice. Nine days following delivery of IL-15 and FL genes, cells were prepared from the indicated organs and stained for human CD45, CD56 plus NKG2D, NKG2A, CD7, CD69, CD94, NKp46, KIR, or CD16. Shown are staining profiles of CD56 versus NKG2D, NKG2A, CD7, CD69, CD94, NKp46, KIR, or CD16 gating on CD45+ human cells. The numbers indicate percentages of cells in the gated region.

FIGS. 10A-10C: Cytotoxicity and stimulation of human NK cells from IL-15 and FL treated humice. (FIG. 10A) NK cells are cytolytic. Nine days after cytokine gene delivery, human NK cells were purified from BM and spleen, mixed at different effector-to-target (E:T) ratios with K562 cells, and cultured for 4 h. Cytolytic activity of NK cells was determined by measuring lactate dehydrogenase enzymatic activity in the supernatant. (FIG. 10B) NK cells produce IFN-γ after poly(I:C) stimulation in vitro. Purified NK cells (5×105) were cultured alone, or in the presence of poly(I:C) (50 μg/ml), or in the presence of poly(I:C) and in vitro differentiated human DCs (5×105) (see Materials and Methods). Supernatants were analyzed 24 h later for human IFN-γ by ELISA. (FIG. 10C) NK cells produce IFN-γ after poly(I:C) stimulation in vivo. Humice were injected intravenously with poly(I:C) (200 pg per mouse). Twenty-four hours after injection, sera were collected and assayed for human IFN-γ by ELISA (n=4). P<0.05.

FIG. 11: Differentiation of human CD34+ cells in vitro. CD34+ human cells were purified from the BM of humice (Left) and cultured in the presence of GM-CSF plus IL-4, or M-CSF for 7 days, or EPO plus IL-3 for 20 days. Cells were then assayed for CD45 plus CD209 and CD11c, or CD14 and CD33, or CD235ab. CD209 versus CD11c and CD14 versus CD33 staining profile are shown for CD45+ cells. CD235ab expression is shown by histograms (bold line). Purified CD34+ cells cultured without EPO and IL-3 was used as controls (thin line).

FIGS. 12A-12C: Human cell proliferation following tetanus toxoid vaccine immunization in humanized mouse. (FIG. 12A) Experimental flow of immunization: On Day 0, 12-week-old humanized mice with similar human leukocyte reconstitution (50-80%) were hydrodynamically injected with plasmids encoding human GM-CSF and IL-4 or blank pcDNA vector (vector). After seven days, these mice were immunized with tetanus toxoid (TT) three times. The first immunization was performed by i.p. injection of 2 1.f. of tetanus toxoid vaccine, followed by another two boosters with two 3-week intervals. The mice were analyzed two weeks after 2nd booster. (FIG. 12B) The spleens from GM-CSF and IL-4 treated mice enlarged significantly. (FIG. 12C) The number of mononuclear cells (MNCs) in the spleens of various humice. Spleens were harvested after immunization. Total splenic MNCs were counted and the number of human CD45⁺ cells was determined by flow cytometry analysis (mean±SEM, n=3 per group).

FIG. 13: Cell surface phenotype of human B cells in GM-CSF and IL-4 treated, TT immunized humice. Cells were prepared from the spleens and stained for human CD45, mouse CD45, CD19 plus IgM, IgD, CD10, CD268, CD5, CD21, CD27, IgG, or CD20. Shown are staining profiles of CD19 versus IgM, IgD, CD10, CD268, CD5, CD21, CD27, IgG, or CD20 gating on CD45⁺ human cells.

FIG. 14: Cell surface phenotype of human T cells in GM-CSF and IL-4 treated, TT immunized humice. Cells were prepared from the spleens and stained for human CD45, mouse CD45, CD19 plus IgM, IgD, CD10, CD268, CD5, CD21, CD27, IgG, or CD20. Shown are staining profiles of CD3 versus T cell activation markers: HLA-DR and CD40L gating on CD45⁺ human cells.

FIGS. 15A-15C: Serum levels of human IgG, IgM and TT specific human IgG in TT immunized humice. Sera were collected from TT immunized humice and measured for human IgG, IgM and TT specific human IgG by ELISA. (FIG. 15A) Human total IgG in the sera. The GM⁻CSF⁺IL-4 treated mice generated a significantly higher human total IgG level than vector treated mice. (FIG. 15B) Human total IgM in the sera. The GM⁻CSF⁺IL-4 treated mice also have higher serum level of total human IgM. (FIG. 15C) Human TT specific IgG in the sera.

FIG. 16A-16B: TT specific human T cell responses in cytokine-treated mice. Two weeks after the third immunization, spleens were harvested and the percentages of human T cells were determined by flow cytometry. For ELISPOT assay, the same number (5×105) of human T cells from different samples were seeded into wells coated with anti-human IFN-65 or anti-human IL-4 antibody and cultured for 24 hrs under three conditions: medium alone (ctrl), in the presence of PMA or in the presence of a TT-specific peptide. ELISPOT was developed. (FIG. 16A) Representative human IFN-γ ELISPOT wells with splenocytes from immunized mice. (FIG. 16B) Representative human IL-4 ELISPOT wells with splenocytes from immunized mice. Data shown are from one of two independent experiments.

FIG. 17: A mixture of DNA plasmids encoding human IL-15, FL, GM-CSF, IL-4 and M-CSF (50 μg each) were dissolved in PBS and injected into 12-week-old humanized mice (n=2). After seven days, the sera were collected and analyzed for these human cytokines by ELISA.

### DETAILED DESCRIPTION OF THE INVENTION

Recently, significant reconstitution of human dendritic cells (DC) and monocytes/macrophages was reported in NOD-scid mice that were engrafted with human fetal thymus, liver, and autologous human CD34⁺ cells (BLT mice) (Wege A K, et al. (2008) Curr Top Microbiol Immunol 324:149-165). Still, human NK cells were absent in BLT mice. As NK cells and myeloid cells play important roles in innate immune responses, development of human non-human mammals, such as humanized mice, with adequate levels of reconstitution of these cell types is critical for realizing the full potential of humanized mouse models in infectious disease research and other research involving blood lineage cells (e.g., hematological disease research such as anemia, immunodeficiencies, cancer).

All blood cell lineages are derived from common human hematopoietic stem cells (HSCs). Cytokines play a key role during their differentiation and maintenance. For example, IL-15 is required for the development and survival of NK cells (Mrozek E, et al. (1996) Blood 87:2632-2640), GM-CSF and IL-4 for dendritic cell (DC) development (Rosenzwajg M, et al. (1996) Blood 87:535-544), macrophage colony stimulating factor (M-CSF) for monocyte/macrophage development and maintenance (Stec M, et al, (2007) J Leukoc Biol 82:594-602), and erythropoietin (EPO) and IL-3 for erythrocyte development (Giarratana M C, et al. (2005) Nat Biotechnol 23:69-74). However, because of evolutionary divergence between human and mouse, these cytokines are species-specific (i.e., the mouse cytokines do not function on human cells). For example, mouse IL-15 has no effect on human NK cells and precursors (Eisenman J, et al. (2002) Cytokine 20:121-129), resulting in poor reconstitution of human NK cells in humice (Huntington N D, et al. (2009) J Exp Med 206:25-34; Kalberer C P, et al. (2003) Blood 102:127-135). Similarly, mouse GM-CSF, IL-4 (Metcalf D (1986) Blood 67:257-267; Mosmann T R, et al. (1987) J Immunol 138:1813-1816), M-CSF (Fixe P, Praloran V (1997) Eur Cytokine Netw 8:125-136), and IL-3 (Stevenson L M, Jones D G (1994) J Comp Pathol 111:99-106) have all been reported not to function on human cells.

Whether poor reconstitution and function of NK cells and myeloid cells in humice are a result of the lack of specific human cytokines was investigated. Described herein are experiments to determine whether expression of human cytokines in the reconstituted mice stimulate differentiation, survival, and function of specific human-blood lineage cells.

This investigation has led to the development of a simple and efficient method to improve the reconstitution of specific human-blood lineage cells in humanized non-human mammals as exemplified using humanized mice. Upon delivery of nucleic acid encoding human IL-15 and Flt-3/Flk-2 ligand (FL), specific human cytokines were detected in the circulation of humice for 2 to 3 weeks. As a result, a significantly elevated number of human NK cells was observed in various organs for more than a month. The cytokine-induced NK cells were fully functional both in vitro and in vivo. Using the same strategy, the reconstitution levels of human dendritic cells, monocytes/macrophages, and erythrocytes were also greatly enhanced in humice. The studies described herein demonstrates that the poor reconstitution of NK cells and myeloid cells in prior models of humanized mice is the result of a lack of appropriate human cytokines required for their differentiation and maintenance, and that delivery (e.g., hydrodynamic delivery) of human cytokine genes is a simple and efficient method to overcome the poor reconstitution of these cell lineages.

Accordingly, in one aspect the invention is directed to a method of reconstituting functional human blood lineage cells (e.g., a single human blood lineage cell (e.g., NK cell); multiple human blood lineage cells (e.g., NK cells, dendritic cells, T cells, B cells etc.) and in some embodiments, all human blood lineage cells) in a non-human mammal. In this embodiment, human hematopoietic stem cells (HSCs) and nucleic acid encoding one or more human cytokines are introduced into the non-human mammal. The non-human mammal is maintained under conditions in which the nucleic acid is expressed and the non-human mammal is reconstituted with the human HSCs in the non-human mammal, thereby reconstituting human hematopoietic stem cells (HSCs) in the non-human mammal.

As used herein, HSCs (e.g., human HSCs) are self renewing stem cells that, when engrafted into a recipient, can “repopulate” or “reconstitute” the hematopoietic system of a graft recipient (e.g., a non-human mammal; an immunodeficient non-human mammal) and sustain (e.g., long term) hematopoiesis in the recipient. HSCs are multipotent stem cells that give rise to (differentiate into) blood cell types including myeloid (e.g., monocytes and macrophages, neutrophils, basophils, eosinophils, erythrocytes, megakaryocytes/platelets, dendritic cells) and lymphoid lineages (e.g., T-cells, B-cells, NK-cells). As shown in the methods described herein, the reconstituted human HSCs can differentiate into human NK cells, human monocytes, human macrophages, human dendritic cells, human red blood cells, human B cells, human T cells or combinations thereof in the non-human mammal.

HSCs express the cell marker CD34 and are commonly referred to as “CD34+”. As understood by those of skill in the art, HSCs can also express other cell markers, such as CD133 and/or CD90 (“CD133+”, “CD90+”). In some instances, HSCs are characterized by markers that are not expressed, e.g., CD38. Thus, in one embodiment of the invention, the human HSCs used in the methods described herein are CD34+, CD90+, CD133+, CD34+CD38−, CD34+CD90+, CD34+CD133+CD38−, CD133+CD38−, CD133+CD9O+CD38−, CD34+CD133+CD90+CD38−, or any combination thereof. In a particular embodiment, the HSCs are both CD34 (“CD34+”) and CD133+(“CD133+”), also referred to herein as “double positive” or “DP” cells or “DPC”. In another embodiment, the HSCs are CD34+CD133+, and can further comprise CD38− and/or CD90+.

HSCs are found in bone marrow such as in femurs, hip, ribs, sternum, and other bones of a donor (e.g., vertebrate animals such as mammals, including humans, primates, pigs, mice, etc.). Other sources of HSCs for clinical and scientific use include umbilical cord blood, placenta, fetal liver, mobilized peripheral blood, non-mobilized (or unmobilized) peripheral blood, fetal liver, fetal spleen, embryonic stem cells, and aorta-gonad-mesonephros (AGM), or a combination thereof.

As will be understood by persons of skill in the art, mobilized peripheral blood refers to peripheral blood that is enriched with HSCs (e.g., CD34+ cells). Administration of agents such as chemotherapeutics and/or G-CSF mobilizes stem cells from the bone marrow to the peripheral circulation. For example, administration of granulocyte colony-stimulating factor (G-CSF) for at least, or about, 5 days mobilizes CD34+ cells to the peripheral blood. A 30-fold enrichment of circulating CD34+ cells is observed with peak values occurring on day 5 after the start of G-CSF administration. Without mobilization of peripheral blood, the number of circulating CD34+ cells is very low, estimated between 0.01 to 0.05% of total mononuclear blood cells.

The human HSCs for use in the methods can be obtained from a single donor or multiple donors. In addition, the HSCs used in the methods described herein can be freshly isolated HSCs, cryopreserved HSCS, or a combination thereof.

As known in the art, HSCs can be obtained from these sources using a variety of methods known in the art. For example, HSCs can be obtained directly by removal from the bone marrow, e.g., in the hip, femur, etc., using a needle and syringe, or from blood following pre-treatment of the donor with cytokines, such as granulocyte colony-stimulating factor (G-CSF), that induce cells to be released from the bone marrow compartment.

The HSCs for use in the methods of the invention can be introduced into the non-human mammal directly as obtained (e.g., unexpanded) or manipulated (e.g., expanded) prior to introducing the HSCs into the non-human mammal. In one embodiment, the HSCs are expanded prior to introducing the HSCs into the non-human mammal. As will be appreciated by those of skill in the art there are a variety of methods that can be used to expand HSCs (see e.g., Zhang, Y., et al., Tissue Engineering, 12(8):2161-2170 (2006); Zhang C C, et al., Blood, 111(7):3415-3423 (2008)). In a particular embodiment, a population of HSCs can be expanded by co-culturing the HSCs with mesenchymal stem cells (MSCs) in the presence of growth factors (e.g., angiopoietin-like 5 (Angplt5) growth factor, IGF-binding protein 2 (IGFBP2), stem cell factor (SCF), fibroblast growth factor (FGF), thrombopoietin (TPO), or a combination thereof) to produce a cell culture. The cell culture is maintained under conditions in which an expanded population of HSCs is produced (e.g., see Maroun, K., et al., ISSCR, 7^{th} Annual Meeting, Abstract No. 1401 (Jul. 8-11, 2009) Attorney Docket No. 4471.1000-001, PCT Application No. PCT/US2010/036664, filed May 28, 2010, published as ______ which is incorporated herein by reference).

In the methods described herein, a (one or more) nucleic acid (e.g., DNA, RNA) encoding one or more human cytokines is also introduced into the non-human mammal to induce differentiation of the human HSCs into functional human cells. As is known in the art, cytokines are proteins that stimulate or inhibit differentiation, proliferation or function of immune cells. Also known in the art are the nucleic acid sequences of numerous human cytokines (see, for example, www.ncbi.nlm.nih.gov). Methods for obtaining nucleic acid encoding one or more cytokines are routine in the art and include isolating the nucleic acid (e.g., cloning) from a variety of sources (e.g., serum), producing the nucleic acid recombinantly or obtaining the nucleic acid from commercial sources.

There are a variety of human cytokines that can be used in the methods of the invention. Examples of such human cytokines include interleukin-12 (IL-12), interleukin-15 (IL-15), Fms-related tyrosine kinase 3 ligand (Flt3L), Flt3L/F1k2 ligand (FL), granulocyte macrophage colony stimulating factor (GM-CSF), interleukin-4 (IL-4), interleukin-3 (IL-3), macrophage colony stimulating factor (M-CSF), erythropoietin (EPO) and a combination thereof Examples of other suitable cytokines for use in the methods described herein are listed in the Table. The type of cytokine and the number of cytokines introduced into the non-human mammal will depend upon which human blood cell lineages are to be reconstituted when differentiation of the human HSCs occur in the non-human mammal. For example, as shown in Example 1, when nucleic acid encoding human IL-15 and Flt-3/Flk-2 ligand was introduced into humanized mice (e.g., by hydrodynamic tail-vein injection), the expression of the human cytokines lasted for 2 to 3 weeks and elevated levels of human NK cells were induced for more than a month. The cytokine-induced NK cells expressed both activation and inhibitory receptors, killed target cells in vitro, and responded robustly to a virus infection in vivo. Similarly, expression of human GM-CSF and IL-4 resulted in significantly enhanced reconstitution of human dendritic cells; expression of macrophage colony stimulating factor resulted in significantly enhanced reconstitution of human monocytes/macrophages; and expression of erythropoietin and IL-3 resulted in significantly enhanced reconstitution of human erythrocytes (see Chen, Q., et al., Proc. Natl. Acad. Sci., USA, 106:21783-21788 (2009) which is incorporated herein by reference). As shown in Example 2, expression of GM-CSF and IL-4 enhanced reconstitution of functional human T cells and human B cells.

In some aspects, at least (comprising) one cytokine, at least 2 cytokines, at least 3 cytokines, at least 4 cytokines, at least 5 cytokines, at least 6 cytokines, at least 7 cytokines, at least 8 cytokine, at least 9 cytokines, at least 10 cytokines, at least 11 cytokines, at least 12 cytokines, at least 13 cytokines, at least 14 cytokines, at least 15 cytokines, at least 16 cytokines, at least 17 cytokines, at least 18 cytokines, at least 19 cytokines, or at least 20 cytokines, are introduced into the non-human mammal. In other aspect, only (consisting, consisting essentially of) one cytokine, 2 cytokines, 3 cytokines, 4 cytokines, 5 cytokines, 6 cytokines, 7 cytokines, 8 cytokine, 9 cytokines, 10 cytokines, 11 cytokines, 12 cytokines, 13 cytokines, 14 cytokines, 15 cytokines, 16 cytokines, 17 cytokines, 18 cytokines, 19 cytokines, or 20 cytokines are introduced into the non-human mammal. Nucleic acid encoding each human cytokine can be introduced simultaneously or sequentially (e.g., in the instances in which more than one cytokine is to be expressed in the non-human mammal, each nucleic acid encoding each cytokine can be introduced in its own single plasmid or vector, or can be introduced in multiple plasmids or vectors; alternatively, all the nucleic acid encoding the cytokines to be introduced can be introduced in a single plasmid or vector).

In the methods of the invention, the HSCs and the nucleic acid encoding one or more cytokines are introduced into a non-human mammal. As used herein, the terms “mammal” and “mammalian” refer to any vertebrate animal, including monotremes, marsupials and placental, that suckle their young and either give birth to living young (eutharian or placental mammals) or are egg-laying (metatharian or nonplacental mammals). Examples of mammalian species that can be used in the methods described herein include non-human primates (e.g., monkeys, chimpanzees), rodents (e.g., rats, mice, guinea pigs), canines, felines, and ruminents (e.g., cows, pigs, horses). In one embodiment, the non-human mammal is a mouse. The non-human mammal used in the methods described herein can be adult, newborn (e.g., <48 hours old; pups) or in utero.

In particular embodiments, the non-human mammal is an immunodeficient non-human mammal, that is, a non-human mammal that has one or more deficiencies in its immune system (e.g., NSG or NOD scid gamma (NOD. Cg-Prkdcscid Il2rgtml Wjl/SzJ) mice) and, as a result, allow reconstitution of human blood cell lineages when human HSCs are introduced. For example, the non-human mammal lacks its own T cells, B cells, NK cells or a combination thereof. In particular embodiments, the non-human mammal is an immunodeficient mouse, such as a non-obese diabetic mouse that carries a severe combined immunodeficiency mutation (NOD/scid mouse); a non-obese diabetic mouse that carries a severe combined immunodeficiency mutation and lacks a gene for the cytokine-receptor γ chain (NOD/scid IL2Rγ−/− mouse); and a Balb/c rag−/− γc−/− mouse.

Other specific examples of immunodeficient mice include, but are not limited to, severe combined immunodeficiency (scid) mice, non-obese diabetic (NOD)-scid mice, IL2rg^{-/-} mice (e.g., NOD/LySz-scid IL2rg^{-/-} mice, NOD/Shi-scid IL2rg^{-/-} mice (NOG mice), BALB/c-Rag^{-/-}IL2rg^{-/-} mice, H2^{d}-Rag^{-/-}IL2rg^{-/-} mice), NOD/Rag^{-/-}IL2rg^{-/-} mice.

In some embodiments, the non-human mammal is treated or manipulated prior to introduction of the human HSCs and the nucleic acid encoding the one or more human cytokines (e.g., to further enhance reconstitution of the human HSCs). For example, the non-human mammal can be manipulated to further enhance engraftment and/or reconstitution of the human HSCs. In one embodiment, the non-human mammal is irradiated prior to introduction of the HSCs and the nucleic acid encoding the one or more cytokines. In another embodiment, one or more chemotherapeutics are administered to the non-human mammal prior to introduction of the HSCs and the nucleic acid encoding the one or more cytokines.

As will also be appreciated by those of skill in the art, there are a variety of ways to introduce HSCs and nucleic acid encoding cytokines into a non-human mammal. Examples of such methods include, but are not limited to, intradermal, intramuscular, intraperitoneal, intraocular, intrafemoral, intraventricular, intracranial, intrathekal, intravenous, intracardial, intrahepatic, intra-bone marrow, subcutaneous, topical, oral and intranasal routes of administration. Other suitable methods of introduction can also include, in utero injection, hydrodynamic gene delivery, gene therapy, rechargeable or biodegradable devices, particle acceleration devises (“gene guns”) and slow release polymeric devices.

The HSCs can be introduced into the non-human using any such routes of administration or the like. In a particular embodiment, the HSCs are injected intracardially into the non-human mammal.

The nucleic acid encoding the one or more cytokines can be also by introduced using any such route of administration as long as the nucleic acid(s) is/are expressed in the non-human mammal. For example, nucleic acid encoding the one or more cytokines can be introduced as naked nucleic acid (naked DNA), in a plasmid (e.g., pcDNA3.1(+)) or in viral vector (e.g., adenovirus, adeno-associated virus, lentivirus, retrovirus and the like). In a particular embodiment, the nucleic acid encoding the one or more cytokines is introduced in a plasmid using hydrodynamic injection (e.g., into tail vein of a non-human mammal).

The HSCs and the nucleic acid encoding the one or more cytokines can be introduced simultaneously or sequentially, and as will be appreciated by those of skill in the art, will depend upon factors, such as the type of non-human mammal being used, the cytokines being expressed and which human blood lineage cells are to be expressed and/or enhanced when differentiation of the human HSCs occur in the non-human mammal. In a particular embodiment, the HSCs are introduced into a newborn pup (e.g., about 48 hours old) and the nucleic acid encoding the cytokines are introduced about 1 month, about 2 months, about 3 months, about 4 months, about 5 months, about 6 months, about 7 months, about 8 months, about 9 months, about 10 months, about 11 months, about 12 months later.

Once the HSCs and the nucleic acid encoding the one or more cytokines are introduced, the non-human mammal is maintained under conditions in which the nucleic acid is expressed and the non-human is reconstituted with the HSCs. Such conditions under which the non-human animals of the invention are maintained include meeting the basic needs (e.g., food, water, light) of the mammal as known to those of skill in the art.

The methods described herein can further comprise determining whether the nucleic acid is expressed, the human HSCs are present and/or the human. HSCs have differentiated into one or more human blood lineage cells. Methods for determining whether the nucleic acid is expressed and/or the non-human is reconstituted with the HSCs are provided herein and are well known to those of skill in the art. For example, flow cytometry analysis using antibodies specific for surface cell markers of human HSCs can be used to detect the presence of human HSCs in the non-human mammal. In addition, sera can be collected from the non-human mammal and assayed for the presence of the human cytokines. Assays for assessing the function of the differentiated HSCs (e.g., NK cells, dendritic cells, T cell, B cells, monocytes/macrophages, erythrocytes) can be also be used. Such assays are also described herein and well known to those of skill in the art. For example, as described herein, cytokine-induced human NK cells killed target cells in an vitro assay (lactate dehydrogenase assay) and responded robustly to a virus infection in vivo.

The ability to reconstitute one or more human blood cell lineages in non-human mammals (e.g., humanized mice) by delivery of nucleic acid encoding one or more human cytokine genes can be used in a variety of ways.

For example, in one aspect, the invention is directed to a method of reconstituting functional human NK cells in a non-human mammal comprising introducing into an immunodeficient non-human mammal human HSCs and nucleic acid encoding one or more human cytokines, wherein the human cytokines promote differentiation of the human HSCs into functional human NK cells when expressed in the non-human mammal. The non-human mammal is maintained under conditions in which the nucleic acid is expressed and the human HSCs differentiate into functional human NK cells in the non-human mammal, thereby reconstituting functional human NK cells in the non-human mammal. In a particular embodiment, the nucleic acid encoding the one or more cytokines encodes human IL-15 and human Flt-3/Flk-2 ligand.

In another embodiment, about 3% to about 25% of leukocytes in the peripheral blood of the non-human mammal are human NK cells (e.g., functional NK cells). In other embodiments, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% of leukocytes in the peripheral blood of the non-human mammal are human NK cells.

In yet other embodiments, expression of human NK cells (e.g., functional NK cells) is maintained (and in some instances, enhanced, compared for example to a suitable control) for about 1 to about 30 days, and in particular embodiments, expression of human NK cells is maintained for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the human NK cells in the non-human mammal express one or more, and in some instances all, of the cell surface markers of the normal (wild type) NK cell found in humans. Such expression indicates that the human NK cells are indeed functional in the non-human mammal. For example in one embodiment, the human NK cells are CD56+ NK cells. In other embodiments, the human NK cells express NKG2D, NKG2A, CD94, KIR, NKp46, CD7, CD69, Cd16, or combinations thereof.

In yet other embodiments, the human NK cells in the non-human mammal capable of killing target cells and expressing IFN-γ upon appropriate stimulation (e.g., a Toll-like receptor 3 agonist poly(I:C); human dendritic cells; adenovirus).

In another aspect, the invention is directed to a method of reconstituting functional human dendritic cells in a non-human mammal comprising introducing into an immunodeficient non-human mammal human HSCs and nucleic acid encoding one or more human cytokines, wherein the human cytokines promote differentiation of the human HSCs into functional human dendritic cells when expressed in the non-human mammal. The non-human mammal is maintained under conditions in which the nucleic acid is expressed and the human HSCs differentiate into functional human dendritic cells in the non-human mammal, thereby reconstituting functional human dendritic cells in the non-human mammal. In a particular embodiment, the nucleic acid encoding the one or more cytokines encodes human GM-CSF and human IL-4. In another embodiment, the nucleic acid encoding the one or more cytokines encodes human GM-CSF, human IL-4 and human Flt-3/Flk-2 ligand.

In other embodiments, the human dendritic cells in the non-human mammal express one or more, and in some instances all, of the cell surface markers of the normal (wild type) dendritic cell found in humans. In one embodiment, the human dendritic cells in the non-human mammal are CD11c+CD209 myeloid dendritic cells (e.g., expressed in the blood, spleen, bone marrow, lung, liver), ILT7+CD303+ plasmacytoid dendritic cells or a combination thereof.

In another aspect, the invention is directed to a method of reconstituting functional human monocytes/macrophages in a non-human mammal comprising introducing into an immunodeficient non-human mammal human HSCs and nucleic acid encoding one or more human cytokines, wherein the human cytokines promote differentiation of the human HSCs into functional human monocytes/macrophages when expressed in the non-human mammal. The non-human mammal is maintained under conditions in which the nucleic acid is expressed and the human HSCs differentiate into functional human monocytes/macrophages in the non-human mammal, thereby reconstituting functional human monocytes/macrophages in the non-human mammal. In a particular embodiment, the nucleic acid encoding the one or more cytokines encodes human macrophage colony stimulating factor.

In other embodiments, the human moncytes/macrophages in the non-human mammal express one or more, and in some instances all, of the cell surface markers of the normal (wild type) moncytes/macrophages found in humans. In one embodiment, the human moncytes/macrophages express CD14+.

In another aspect, the invention is directed to a method of reconstituting functional human erythrocytes in a non-human mammal comprising introducing into an immunodeficient non-human mammal human HSCs and nucleic acid encoding one or more human cytokines, wherein the human cytokines promote differentiation of the human HSCs into functional human erythrocytes when expressed in the non-human mammal: The non-human mammal is maintained under conditions in which the nucleic acid is expressed and the human HSCs differentiate into functional human erythrocytes in the non-human mammal, thereby reconstituting functional human erythrocytes in the non-human mammal. In a particular embodiment, the nucleic acid encoding the one or more cytokines encodes human erythropoietin and IL-3.

In other embodiments, the human erythrocytes in the non-human mammal express one or more, and in some instances all, of the cell surface markers of the normal (wild type) erythrocytes found in humans. In one embodiment, the human erythrocytes express CD235ab+.

In yet other embodiments, the human erythrocytes in the non-human mammal comprise about 1% to about 10%, or about 3% to about 5%, of all red blood cells in the non-human mammal. In particular embodiments, the human erythrocytes in the non-human mammal comprise about 1%, about 2%, about 3%, about 4%, about 5%, about 6%, about 7%, about 8%, about 9% or about 10% of all red blood cells in the non-human mammal.

In a particular aspect, the invention is directed to a method of reconstituting functional human T cells and human B cells in a non-human mammal comprising introducing into an immunodeficient non-human mammal human HSCs and nucleic acid encoding one or more human cytokines, wherein the human cytokines promote differentiation of the human HSCs into functional human T cells and human B cells when expressed in the non-human mammal. The non-human mammal is maintained under conditions in which the nucleic acid is expressed and the human HSCs differentiate into functional human T cells and human B cells in the non-human mammal, thereby reconstituting functional human T cells and human B cells in the non-human mammal. In one embodiment, the nucleic acid encoding the one or more cytokines encodes GM-CSF and IL-4. The method can further comprise immunizing the non-human mammal with an immunogen, and maintaining the non-human animal under conditions in which the non-human mammal produces human antibodies directed against the immunogen.

In yet another particular aspect, the invention is directed to a method of generating human antibodies directed against an immunogen in a non-human mammal. In this method human hematopoietic stem cells (HSCs) and nucleic acid encoding one or more human cytokines, wherein the human cytokines promote differentiation of the human HSCs into functional human T cells and human B cells, are introduced into the non-human mammal. The non-human mammal is maintained under conditions in which the nucleic acid is expressed and the HSCs differentiate into functional human T cells and human B cells in the non-human mammal. The non-human mammal is immunized with the immunogen, and maintained under conditions in which the human B cells produce human antibodies directed against the immunogen in the non-human mammal, thereby generating human antibodies directed against the immunogen in the non-human mammal. In one embodiment, the nucleic acid encoding the one or more cytokines encodes GM-CSF and IL-4.

As is known in the art, an “immunogen” is a substance capable of inducing an immune response and promoting antibody production. A variety of immunogens for use in the methods are known in the art. For example, the immunogen can be all or an immunogenic portion of: a protein from a human or other species, a cell surface protein (e.g., of normal or diseases cells, such as tumor cells), an organism (e.g., immunogenic portions of an organism include coats, capsules, cell walls, flagella, fimbrae, and toxins of an organism), a viral protein, a bacterial protein, a toxin, a polysaccharide, a lipoprotein, a modified protein (e.g., acetylated, methylated, glycosyated), a nucleic acid (e.g., DNA, RNA when combined with a peptide, protein or polysaccharide), a chemical epitope, or the like.

These methods can further comprise isolating human B cells that produce the human antibodies from the non-human mammal. Methods for isolating B cells from a non-human mammal are known in the art. For example, cell sorting using flow cytometry or magnetic purification based on antibodies specific for B cell specific proteins (e.g., see Current Protocols in Immunology, Copyright© 2010 by John Wiley and Sons, Inc. ed. John E. Coligan et al.).

As is known in the art, an “antibody” or “immunoglobulin” is a protein component of the immune system produced by B cells that circulates in the blood, recognizes immunogens like bacteria and viruses, and neutralizes them. After exposure to an immunogen, antibodies continue to circulate in the blood, providing protection against future exposures to that antigen. Any type of antibody produced by human B cells can be obtained using the methods described herein. The monoclonal antibodies can be polyclonal or monoclonal antibodies. Examples of such antibodies are well known in the art and include IgG (e.g., IgG1, IgG2, IgG3, IgG4), IgM, IgD and IgA.

The methods can further comprise contacting the isolated human B cells with immortalized cells, thereby producing a combination; and maintaining the combination under conditions in which the human B cells and the immortalized cells fuse to form a hybridoma that produces monoclonal antibodies directed against the immunogen.

As is known in the art, at an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)), the EBV-hybridoma technique (Cole et al., Monoclonal Antibodies and Cancer Therapy, Alan R. Liss, Inc., pp. 77-96 (1985)) or trioma techniques. The technology for producing hybridomas is well known (see generally Current Protocols in Immunology, Coligan et al., (eds.) John Wiley & Sons, Inc., New York, N.Y. (1994)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody directed against an immunogen (see, e.g., Current Protocols in Immunology, supra; Galfre et al., Nature, 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)).

Moreover, the ordinarily skilled worker will appreciate that there are many variations of such methods as well as other methods that can be used to obtain the antibody produced by the human B cells. For example, the sequence that encodes all or a functional portion of the human antibodies expressed by the human B cells can be cloned using known techniques. Typically, the approach involves isolating antigen-specific B cells (e.g., stained with fluorochrome-labeled antigen, sortinghy flow cytometry) and amplification of a VDJ portion of the antibody gene using degenerate primers and single cell polymerase chain reaction (PCR). The cloned and sequenced VDJ portion of the antibody gene are combined with the constant region gene segments to produce antibody in cell lines such as a CHO cell line (e.g., see Hahn, S., et al., Cell Mol. Life Sci. (2000), 57(1):96-105).

Another example of a method for producing and/or isolating the human antibodies produced by the non-human mammal comprises virally immortalizing the human B cells. In this method, for example, Epstein-Barr Virus (EBV) or modified EBV can be used to immortalize B cells (e.g., see Lanzavecchia, A., Curr. opin. Biotechnol. (2007) 18(6):523-528).

As will be appreciated by one of skill in the art, in addition to cytokines, expression of other proteins (e.g., human proteins; human secreted proteins), such as growth factors, steroids, and/or small molecules, can be used in the methods to improve reconstitution and/or function of human cells beyond blood lineage cells. For example, an agonist of one or more of the human cytokines can be introduced into the non-human mammal to enhance reconstitution of the HSCs.

As will be appreciated by one of skill in the art, “functional (or “biologically active” or “mature”) human NK cells”, “functional human dendritic cells”, “functional human monocytes/macrophages”, “functional human erythrocytes, “functional human T cells” and “functional human B cells” all refer to the fact that the differentiated cells (whether human NK cell, human dendritic cells, human monocytes/macrophages, human erythrocytes, human T cell, or human B cells) express one or more, and in some instances all, of the cell surface markers of the corresponding normal (wild type) cell found in humans, and as a result, function similarly in the non-human mammal as they function in a human.

Assays for determining the function of human blood lineage cells in the non-human mammal are known to those of skill in the art and are described herein. For example, an NK cytotoxicity assay can be used to determine the function of the NK cells in the non-human mammals.

In certain aspects of the invention, reconstitution of human blood cell lineages and/or a particular human cell lineage (e.g., NK cell, dendritic cell, monocytes/macrophages, erythrocytes, T cells, B cells) is enhanced in the non-human mammal. Enhanced reconstitution refers to, for example, an enhanced expression of the cell type (e.g., an increase in number of the one or more human blood lineage cell; an increase in time the cell type is expressed (e.g., >30 days)) compared to a suitable control. Such controls are apparent to those of skill in the art. An example of a suitable control is a non-human mammal to which human HSCs, but not nucleic acid encoding one or more cytokines, had been introduced.

Other aspects of the invention include compositions. In one aspect, the invention encompasses non-human animals produced by the methods described herein.

In other aspects, the invention encompasses hybridomas (isolated hybridomas) produced by the methods described herein and monoclonal antibodies (isolated monoclonal antibodies) produced by the hybridomas.

As used herein, “isolated” (e.g., “isolated B cells”; “isolated hybridomas”, “isolated monoclonal antibodies”) refers to substantially isolated with respect to the complex (e.g., cellular) milieu in which it naturally occurs, or organ, body, tissue, blood, or culture medium. In some instances, the isolated material will form part of a composition (for example, a crude extract containing other substances), buffer system, culture system or reagent mix. In other circumstances, the material can be purified to essential homogeneity. An isolated B cell population can comprise at least about 50%, at least about 80%, at least about 85%, at least about 90%, at least about 95%, or at least about 99% (on a total cell number basis) of all cells present. In one embodiment, the invention is directed to isolated, or substantially isolated (or purified, substantially purified) B cells, hybridomas and/or monoclonal antibodies produced by the methods described herein.

Thus, as shown herein, the primary reason for the poor human innate and adaptive responses observed in humanized mice is due to a low level reconstitution of specific human blood lineage cells, and/or poor functional maturation of specific human blood cell lineages, because of a lack of proper human cytokine expression in reconstituted mice. Described herein is an efficient and versatile method to express various human cytokines in reconstituted non-human mammals (e.g., mice) and significantly improve the reconstitution of human blood lineage cells in the resulting non-human mammal.

To express human cytokines in the non-human mammal, DNA vectors encoding human cytokines were introduced into the mammal using hydrodynamic injection (e.g., 10% body weight in 7 seconds). In this embodiment, some of the injected DNA was taken up by hepatocytes, resulting in expression of human cytokine in mice. By introducing nucleic acid encoding human cytokines in this manner, development of specific human cell subsets has been improved, resulting in increased numbers of human cells and immune responses.

Specifically, human cytokines were cloned into a vector. Engineered human interleukin 15 (IL-15) and Fms-related tyrosine kinase 3 ligand (Flt3L) genes were cloned into pcDNA3.1(+) vector individually.

pcDNA3.1(+) plasmid was used as the vector for in vivo gene delivery. For NK cell induction in vivo, pcDNA-IL2/IL15 and pcDNA-Flt3L plasmids were constructed. In brief, the signal peptide sequence of human IL-15 was replaced by that of human IL-2 because the signal peptide of IL-15 is unusually long (48 aa) and it limits the secretion of IL-15. Early-acting cytokine flt3 ligand (FL) was used to increase the frequency of NK cell precursors responding to IL-15. These two recombinant gene sequences were inserted to pcDNA3.1(+) vector.

Plasmid was administered to mice via rapid injection of a large amount of solution through the tail vein by a hydrodynamics-based gene transfer technique. Briefly, 8 to 12 week-old humanized mice were intravenously injected with 50 μg of each plasmid in 1.8 ml saline within 7 s.

These mice were bleed through tail vein on Day 7 post injection to primarily analyze the reconstitution of human immune cells in blood. Then some mice were sacrificed on Day 9 and Day 16 to analyze the dynamics of human cell reconstitution in liver, lung, spleen, bone marrow, and lymph nodes. Sera were collected on different time points to analyze the human cytokine levels in circulating blood by ELISA.

Following hydrodynamic injection, significant levels of IL-15 and Flt3L were detected in the serum for 2-3 weeks. Correspondingly, reconstitution of CD56⁺CD3⁻ human natural killer (NK) cells in blood, spleen, bone marrow, lung and liver was significantly increased to levels comparable to those observed in human organs. The absolute number of CD45⁺ human cells in all the organs also increased.

The NK cells generated by this method in humanized mice had normal NK cell phenotypes and were functional with respect to interferon-γ (IFN-γ) production in response to polyl:C and LPS stimulation both in vitro and in vivo. NK cells purified from humanized mice showed cytotoxic activity against K562 target cells in vitro. Furthermore, NK cells responded to virus infection in vivo. Using a similar approach, dendritic cell reconstitution was enhanced by expressing GM-CSF and IL-4, macrophages and monocytes reconstitution was enhanced by expressing MCSF and human red blood cell reconstitution was enhanced by expressing EPO and IL3.

Hydrodynamic injection of naked DNA resulted in the detection of human cytokines in the serum for 2-3 weeks. As is apparent to those of skill in the art, human genes can also be expressed via viral vectors (e.g., adenovirus-mediated gene expression; lentivirus-mediated gene expression), which can result in high level and prolonged human gene expression in mice.

The reconstitution of some kinds of human immune cells are very low in NOD/SCID IL2Rγ^{-/-} mice transplanted with human hematopoietic stem cells because of the poor reactivity to mouse cytokine environment. Many cytokines which are essential for immune cell development and maturation show species-specific activities like IL-15 to NK cell, GM-CSF/IL-4 to DC, M-CSF to macrophage and so on. Described herein is a hydrodynamics-based in vivo transfection procedure utilizing administration of naked cytokine expression plasmids that resulted in significant high levels of systemic exogenous human cytokine expression to promote human immune cell development in humanized mice.

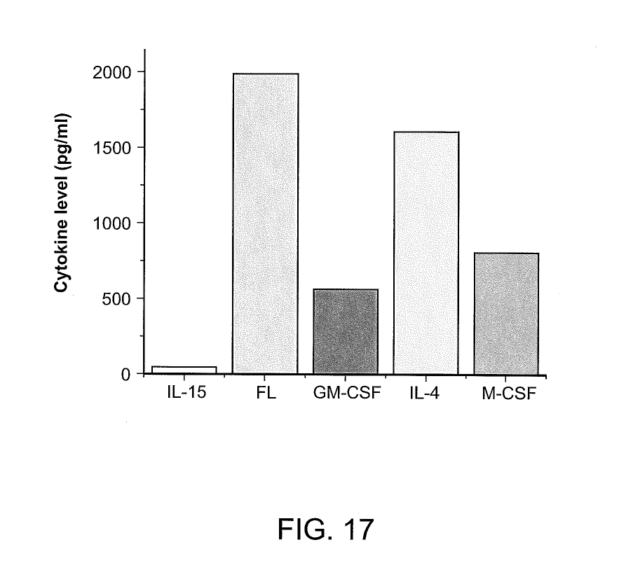

The methods and compositions provide numerous advantages over current methods used to reconstitute non-human mammal with blood cell lineages from human HSCs. For example, the nucleic acid encoding the cytokine need only be introduced once to achieve the desired result; use of nucleic acid encoding cytokines is much easier for large-scale preparation, much more stable for long-term storage and much more convenient for genetic engineering; hydrodynamic-based injections can be conducted in a 3-week-long systemic human cytokine expression, and it is likely that the expression can last longer using adenoviral and/or lentiviral vectors to introduce the cytokine; multiple gene constructs can be combined together (see FIG. 17); and the methods provide a simple and efficient method to reconstitute HSCs (e.g., myeloid cells) in the humanized mice.

### EXEMPLIFICATION

### Example 1

### Expression of Human Cytokines Dramatically Improves Reconstitution of Specific Human-Blood Lineag Cells in Humanized Mice (Humice)

### Materials and Methods

HSC Isolation, Construction of Humanized Mice, and Hydrodynamic Gene Delivery. Human cord blood was obtained from Singapore Cord Blood Bank. Cord blood mononuclear cells (MNCs) were separated by Ficoll-Hypaque density gradient. CD34+ cells were purified with the RosetteSep® system according to the manufacturer's protocol (Stein Cell Technologies). The purity of CD34⁺ cells was >95%. To expand HSCs, purified CD34⁺ cells were cultured for 11 to 14 days in serum-free medium in the presence of defined factors (Zhang C C, Kaba M, Iizuka S, Huynh H, Ladish H F (2008) Blood 111:3415-3423). Both unexpanded and expanded HSCs were used to generate humanized mice.

NSG mice were purchased from the Jackson Laboratories and maintained under specific pathogen-free conditions in the animal facilities at Nanyang Technological University and National University of Singapore. To reconstitute mice, newborn pups (less than 48 h old) were irradiated with 100 cGy using a Gamma radiation source and injected intracardially with CD34⁺CD133⁺ cells (1×105 cells/recipient). Human cytokine genes were cloned separately into pcDNA3.1(+) vector (Invitrogen). Plasmid DNA was purified by Maxi-prep Kit (Qiagen). For hydrodynamic gene delivery, 12-week old humice were injected with 50 μg of each plasmid in a total of 1.8-ml saline within 7 s using a 27-gauge needle. All research with human samples and mice was performed in compliance with the institutional guidelines of the National University of Singapore and Nanyang Technological University.

Single Cell Preparation, Antibodies, and Flow Cytometry. Single-cell suspensions were prepared from spleen and bone marrow (BM) by standard procedures. To isolate MNCs from humice liver, the liver was pressed through a 200-gauge stainless steel mesh and debris was removed by centrifugation at 50×g for 5 min. Supernatants containing MNCs were collected, washed in PBS, and resuspended in 40% Percoll (Sigma) in RPMI medium 1640. The cell suspension was gently overlaid onto 70% Percoll and centrifuged at 750×g for 20 min. MNCs were collected from the interphase, washed twice in PBS. To isolate MNCs from the lung, the lung was minced, suspended in medium containing 0.05% collagenase (Sigma) and 0.01% DNase I (Sigma), and incubated at 37° C. for 20 min. The lung samples were passed through a 200-gauge stainless steel mesh, and MNCs were isolated with Percoll centrifugation as described above.

The following antibodies were used: CD3 (SK7), CD34 (581), CD19 (HIB19), NKG2D (1D11), NKp46 (9E2), CD94 (DX22), CD16 (3G8), CD56 (B159), HLA-DR (L243), CD14 (M5E2), CD11c (B-1y6), CD209 (DCN46), CD7 (M-T701), CD45 (2D1), CD69 (L78), CD33(WM53) from Becton-Dickson; KIR2DL2/L3 (DX27), ILT7 (17G10.2) and CD235ab (HIR2) from BioLegend; CD303 (AC144) from Miltenyi Biotec; CD159a (NKG2A; Z199) from Beckman Coulter; and CD133 (EMK08) and mouse CD45.1 (A20) from eBioscience. Cells were stained with appropriate antibodies in 100-μl PBS containing 0.2% BSA and 0.05% sodium azide for 30 min on ice. Flow cytometry was performed on a LSRII flow cytometer using the FACSDiva software (Becton, Dickinson and Co.). Ten thousand to 1,000,000 events were collected per sample and analyzed using the Flowjo software.

Differentiation of Human CD34+ Cells in Vitro. BM MNCs were isolated from 12-week-old humice. CD34+ cells were enriched by MACS® microbeads (Miltenyi Biotec). Purified cells were culture in RPMI 1640, 10% FCS at 37° C. and 5% CO₂. For the differentiation of NK cells, DCs, monocytes/macrophages, and erythrocytes, 50 ng/ml SCF, 50 ng/ml FL and 50 ng/ml IL-15; 50 ng/ml SCF, 20 ng/ml GM-CSF and 50 ng/ml IL-4; 50 ng/ml SCF and 30 ng/ml M-CSF; and 100 ng/ml SCF, 5 ng/ml IL-3 and 3 U/ml EPO were used, respectively. All of the cytokines were purchased from R&D Systems.

NK Cell Cytotoxicity Assay and Stimulation. Nine days after gene delivery, CD56⁺ NK cells were purified from spleen and BM by positive selection using the Stem cell PE selection Kit (Stern Cell Technologies). Cells were washed and resuspended in IMDM containing 2% FCS, and cytotoxicity against the NK-sensitive target K562 (ATCC) was determined in a 4-h lactate dehydrogenase release assay (CytoTox 96; Promega).

For in vitro stimulation, purified NK cells were cultured in RPMI 1640, 10% FCS, 2-mM L-glutamine, 1-mM sodium pyruvate, penicillin, and streptomycin, either with or without human DCs, at 37° C. and 5% CO₂ for 24 h. Human DCs were differentiated from cord blood CD34⁺ cells as described (Rosenzwajg M, Canque B, Gluckman J C (1996) Blood 87:535-544). Next, 50 pg/ml poly(I:C) (Sigma) was added into the culture to stimulate NK cells in vitro. For in vivo stimulation, humice were i.v. injected with 200 μg poly(I:C). IFN-γ levels in the serum or in the culture supernatants were measured with ELISA Kits (R&D Systems).

Adenovirus Infection, ALT, and Histology. The replication-deficient, E1 and E3-deleted, type 5 Adeno-X virus expressing green fluorescent protein (AdGFP) was purchased from Clontech. AdGFP were propagated in HEK293 cells and purified by CsCl discontinued density gradient centrifugation. Humice were challenged with 4×10⁹ pfu AdGFP viruses by hydrodynamic injection through the tail vein. Five days after adenovirus infection, sera were collected and analyzed for ALT activities using cobas c 111 analyzer (Roche Diagnostics Ltd.).

For histological analysis, the livers were removed, embedded in paraffin and 5-μm-thick sections were prepared. The paraffin sections were stained with H&E and analyzed via a light microscope. For two-color immunofluorescence staining, after blocking of nonspecific staining, deparaffinized sections were stained with optimal dilutions of PE-conjugated anti-human CD56 antibody (MEM-188; Biolegend). Sections were analyzed with MIRAX MIDI Fluorescence microscope (Zeiss).

Statistical Analysis. Data are presented as mean and standard error of the mean. Differences between groups were analyzed via Student t-test. A P-value of <0.05 was considered statistically significant. All calculations were performed using the Origin 8.0 software package.

### Results

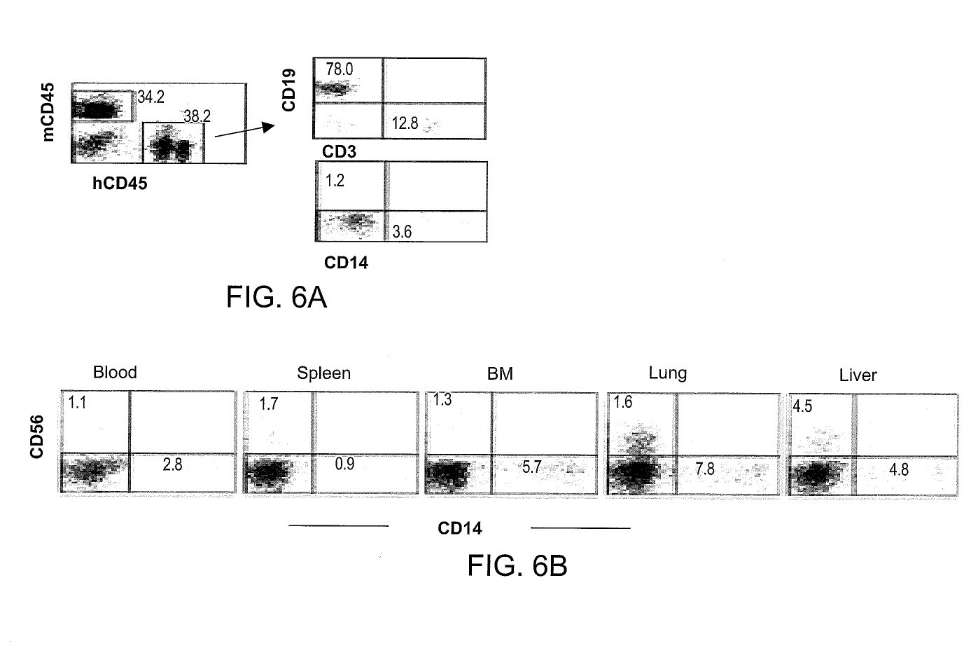

Stimulation of NK Cell Differentiation by Human Cytokines in Vitro. To construct humanized mice, CD34− HSCs isolated from human cord blood were adoptively transferred into sublethally irradiated NSG pups. Twelve weeks after reconstitution, mononuclear cells (MNCs) from peripheral blood were stained with antibodies specific for human CD45 and mouse CD45 (FIG. 6A-6B). The average reconstitution rate was ˜50% in the blood [reconstitution rate=% CD45⁺ human cell/(% CD45⁺ human cell+% CD45⁺ mouse cell)]. Among the CD45⁺ human leukocytes, the level of CD19⁺ B cells ranged from 40 to 85% and the level of CD3⁺ T cell ranged from 10 to 50%. Although NK cells were detected in the blood, BM, spleen, lung, and liver, their frequency was significantly lower than that in the corresponding human tissues or mouse tissues (see FIGS. 6A-6B).

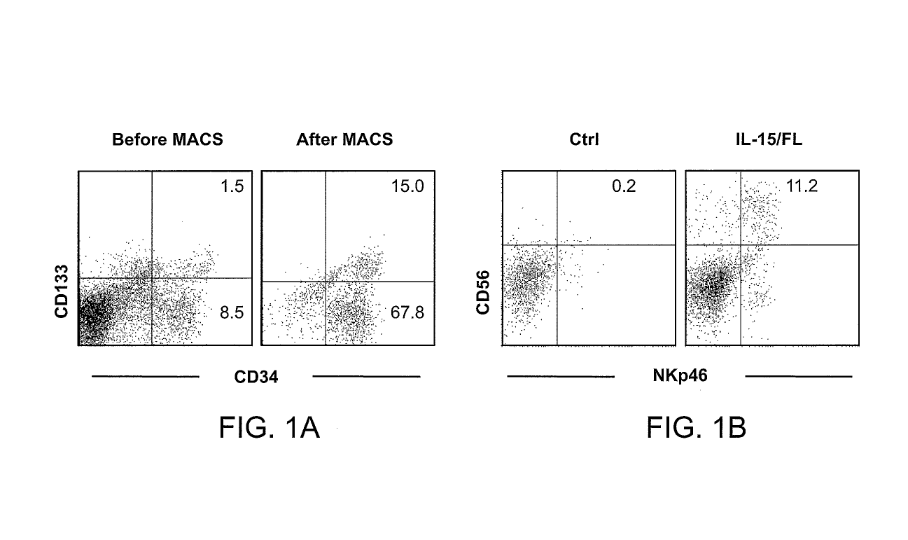

To determine the cause underlying the poor NK cell reconstitution in humice, we tested whether human CD34⁺ cells from the BM of humice can be stimulated by human IL-15 and FL to differentiate into NK cells in vitro. FL stimulates differentiation of multiple hematopoietic cell lineages, including CD34⁺ NK progenitors that can respond to IL-15 (Yu H, et al. (1998) Blood 92:3647-3657). The combination of FL and IL-15 is expected to favor the differentiation of CD34⁺ precursors toward NK cells. Thus, purified human CD34⁺ cells (>80%) (FIG. 1A) from humice BMs were cultured for 7 days in the presence of FL and IL-15 and analyzed for expression of NK cell markers CD56 and NKp46. In the presence of the cytokines, ˜11% of cells were positive for both CD56 and NKp46, whereas very few cells were positive in the absence of the cytokines (FIG. 1B). These results suggest that CD34⁺ human cells in the BM of humice are capable of differentiating into NK cells if the appropriate cytokine environment is provided.

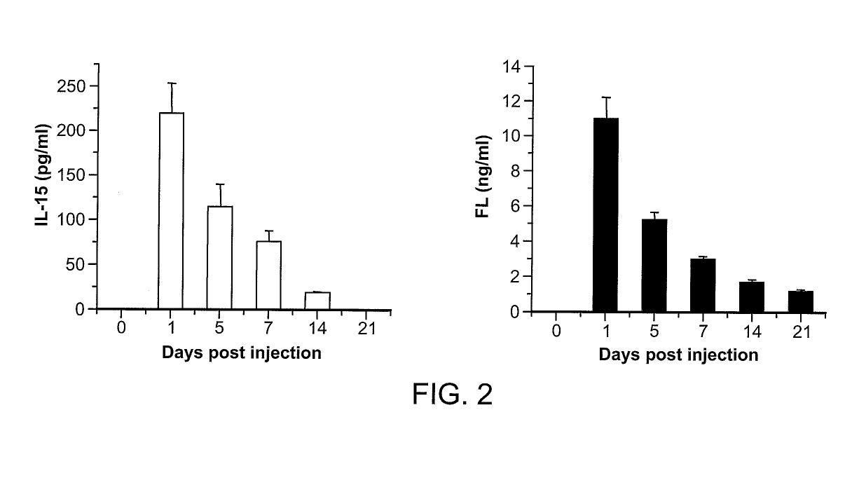

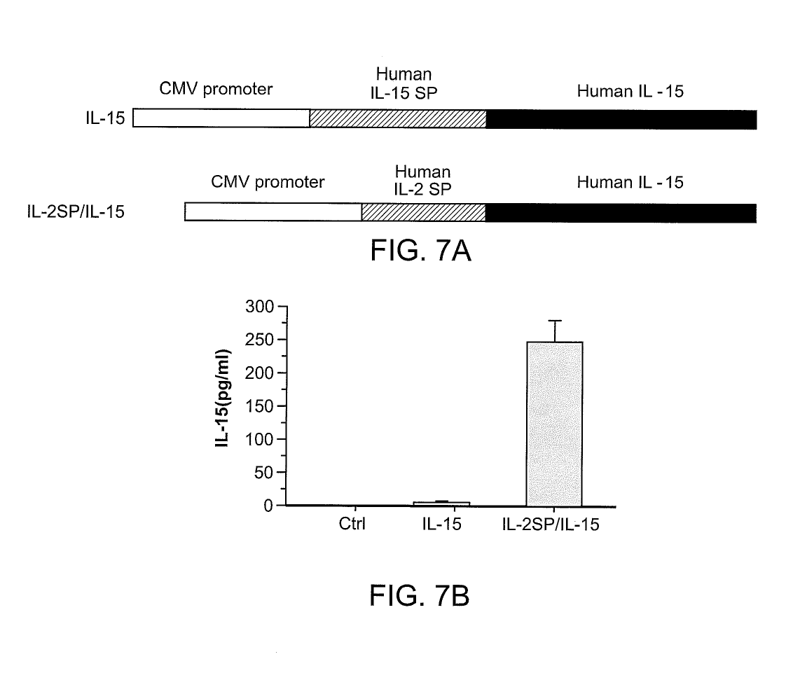

Expression of Human Cytokines in Mice by Hydrodynamic Injection of Plasmid DNA. The finding that human NK cells developed in vitro in the presence of IL-15 and FL suggests that these human cytokines might also stimulate NK cell development in humice. One way to introduce human cytokines into mice is by daily injection of recombinant proteins. Because this way is cumbersome and expensive, we expressed human cytokines in mice by hydrodynamic delivery of cytokine-encoding DNA plasmid. Human IL-15 has an unusually long signal peptide sequence (45 aa residues), which is known to lead to poor secretion of IL-15 (Meazza R, et al. (1997) Eur J Immunol 27:1049-1054). To increase the level of IL-15 secretion, we constructed an IL-15-expressing vector in which the IL-15 signal peptide was replaced by the signal peptide of human IL-2 (FIGS. 7A-7B). This replacement increased the serum level of IL-15 ˜100-fold (FIG. 7B). With a single hydrodynamic injection of 50 μg IL-15-encoding plasmid, a high level of IL-15 was detected in the serum 1 day after injection and a significant level was maintained for 14 days (FIG. 2). Similarly, a single injection of FL-encoding plasmid resulted in expression of FL in the serum for 21 days. Thus, hydrodynamic delivery of cytokine genes is a simple and efficient method to introduce human cytokines in mice.

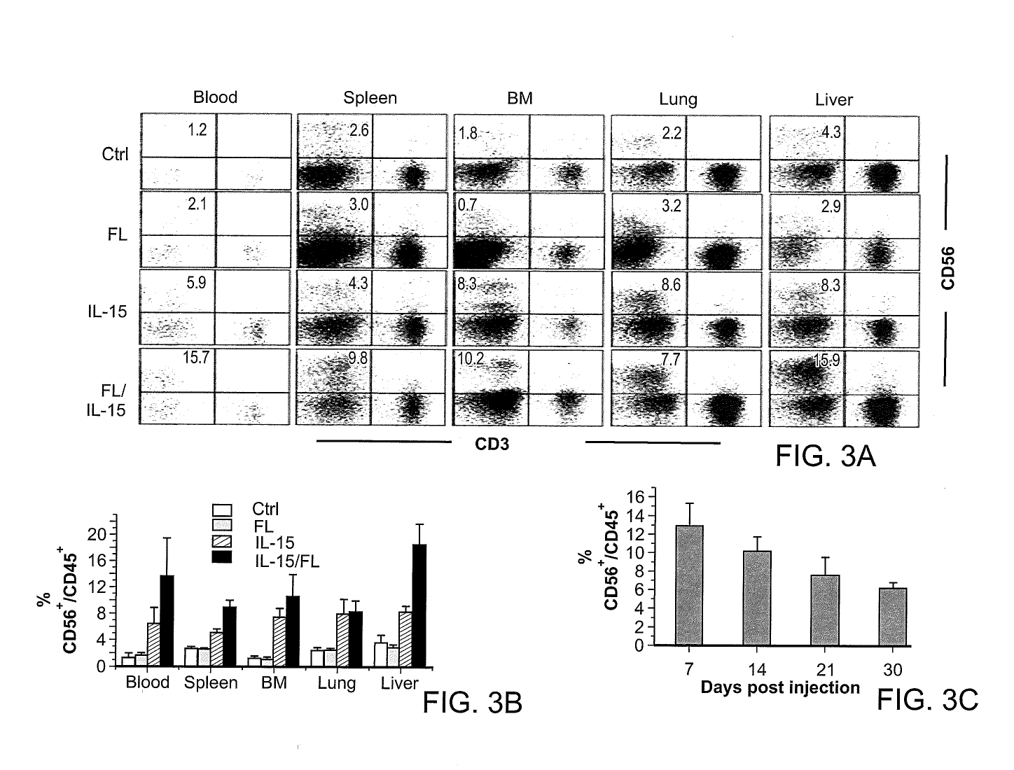

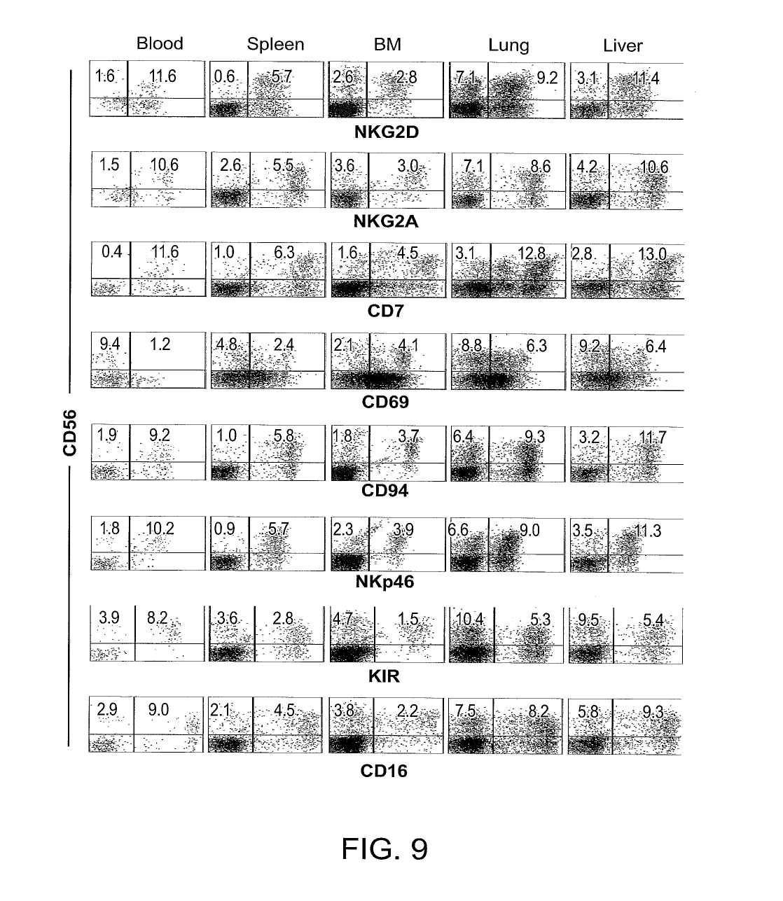

Enhanced Reconstitution of Human NK Cells Following IL-15 and FL Gene Delivery. To determine the effect of IL-15 and FL expression on NK cell development, 9 days after gene delivery humice were analyzed for NK cell reconstitution in various organs by flow cytometry. Injection of empty pcDNA vector or FL-encoding vector did not significantly affect the frequency of CD56⁺ NK cells (FIG. 3A). However, expression of IL-15 significantly increased the frequency of CD56⁺ NK cells in the blood, spleen, BM, lung, and liver (FIGS. 3A and 3B). The increase in frequency of NK cells was even more dramatic when both IL-15 and FL were expressed in humice, reaching the level observed in normal human peripheral blood (5% -21% of leukocytes) (Maurice R G, O'Gorman ADD (2008) Handbook of Human Immunology (CRC Press, Boca Raton)) and normal mouse tissues (Zhang J, et al. (2005) Cell Mol Immunol 2:271-280). Corresponding to the increased frequency of NK cells, the absolute numbers of NK cells were markedly increased in the spleen and BM (Fig. S3B). Furthermore, the elevated frequency of CD56+ NK cells in the blood was maintained for at least 30 days after gene delivery (FIG. 3C). In addition, cytokine-induced NK cells expressed many of cell surface receptors known to be important for NK cell function (FIG. 9), including the activating receptor NKG2D, inhibitory receptors NKG2A, CD94, and KIR, the natural cytotoxicity triggering receptor NKp46, the NK cell marker CD7, the early activation marker CD69, and the FC receptor CD16. These results indicate that cytokine-induced NK cells exhibit the characteristic surface phenotype of normal NK cells.

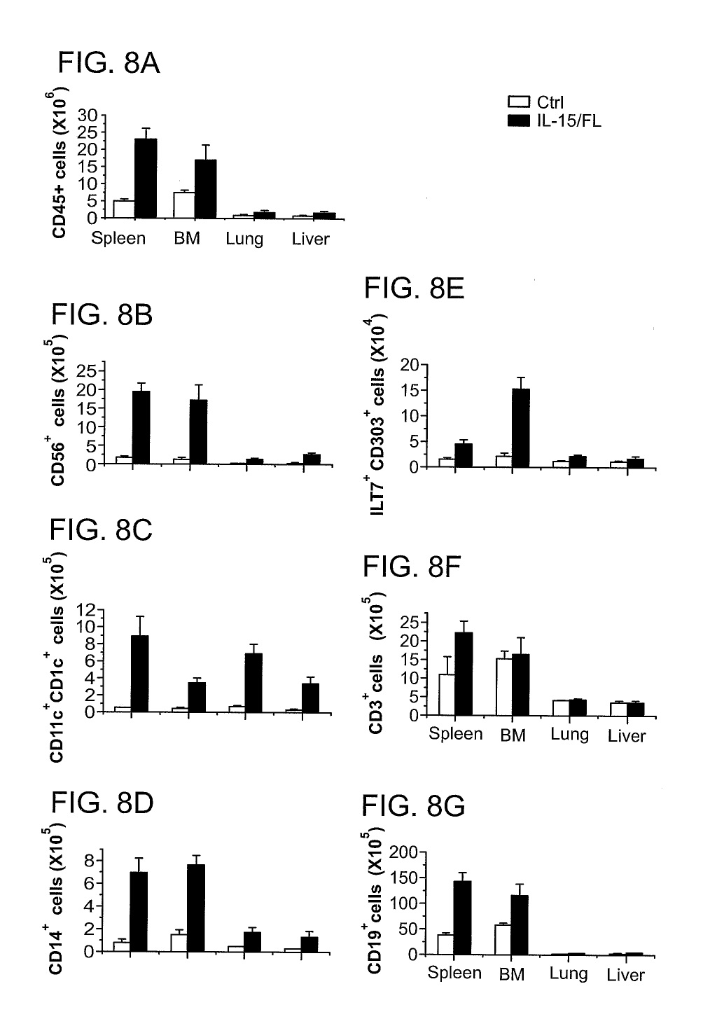

In addition to stimulating NK cell development, both FL and IL-15 are known to exert effect on other hematopoietic cell lineages (Diener K R, Moldenhauer L M, Lyons A B, Brown M P, Hayball. J D (2008) Exp Hematol 36:51-60; Dong J, McPherson C M, Stambrook P J (2002) Cancer Biol Ther 1:486-489; Blom B, Ho S, Antonenko S, Liu Y J (2000) J Exp Med 192:1785-1796; Armitage R J, Macduff B M, Eisenman J, Paxton R, Grabstein K H (1995) J Immunol 154:483-490). Thus, cells from spleen, BM, lung, and liver of humice were enumerated and analyzed by flow cytometry. Expression of IL-15 and FL also induced significant increase in CD 14⁺ monocytes/macrophages, CD11c⁺CD1c⁺ myeloid dendritic cells, ILT7⁺CD303⁻ plasmacytoid dendritic cells, and CD19⁺ B cells in the spleen and BM (see FIGS. 8A-8G). These results demonstrate that expression of human IL-15 and FL dramatically improves the reconstitution of NK cells as well as other myeloid and lymphoid cells in humanized mice.

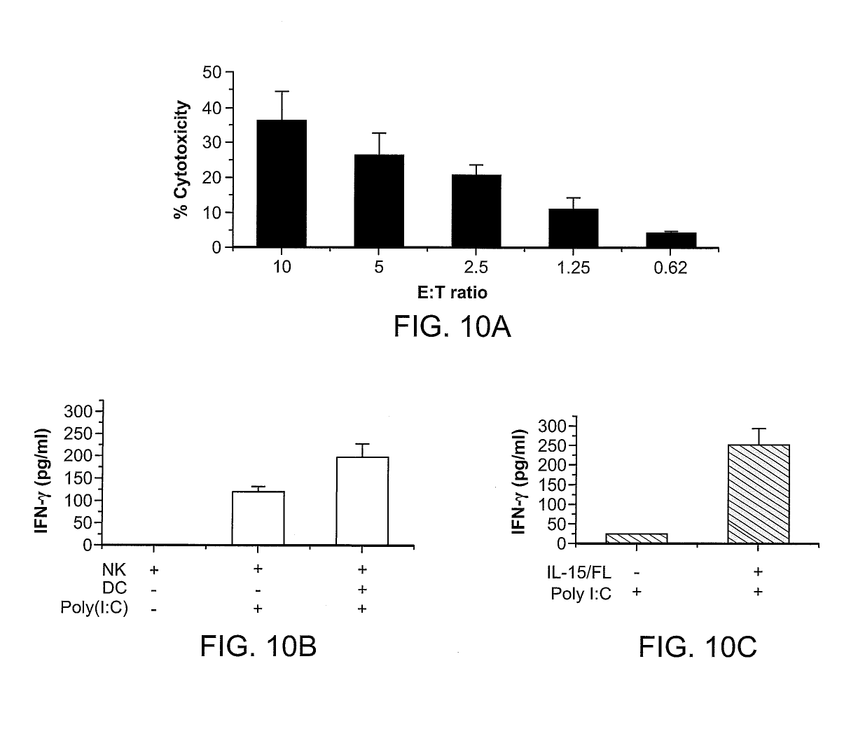

Cytokine-Induced NK Cells are Functional. We investigated whether cytokine-induced human NK cells are functional (i.e., able to kill target cells and express IFN-γ following appropriate stimulation). CD56⁺ NK cells were purified from the BM and spleen of IL-15- and FL-treated mice. When mixed with MHC class I-deficient target cells K562, we observed an increased level of target cell lysis with increasing numbers of NK cells added (FIG. 10A). When purified NK cells were stimulated with a Toll-like receptor 3 agonist poly(I:C), which is known to activate NK cells to produce proinflammatory cytokines (Schmidt K N, et al. (2004) J Immunol 172:138-143), IFN-γ was detected in the culture supernatant (FIG. 10B). In the presence of human DCs, the level of IFN-γ secretion was further increased. When poly(I:C) was injected into humanized mice, a significantly increased level of IFN-γ was detected in the serum of humice that were injected with cytokine-encoding DNA compared to the noninjected humice (FIG. 10C).

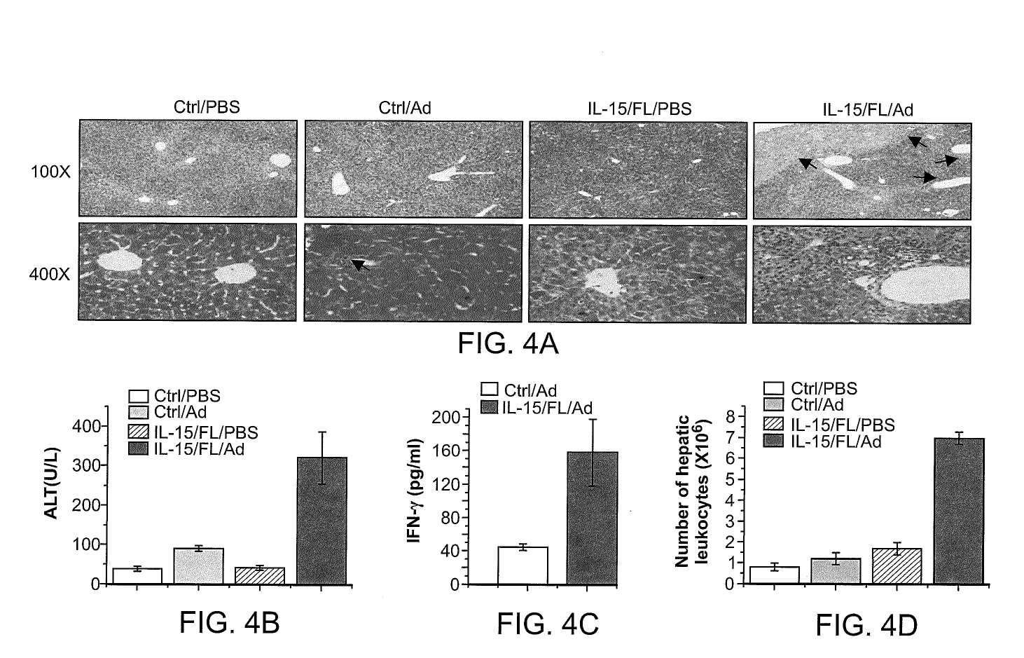

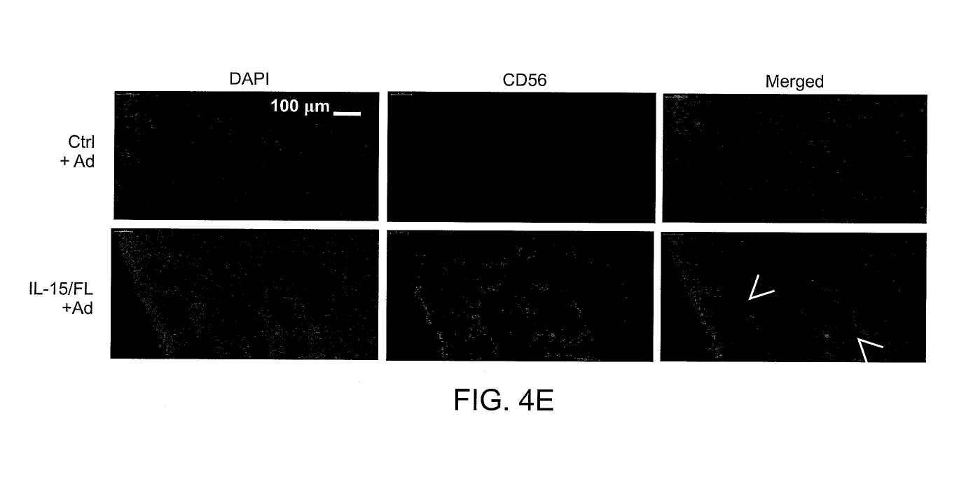

We also challenged humice with adenovirus, which is known to cause NK cell-dependent liver damage (Chen Q, Wei H, Sun R, Zhang I, Tian Z (2008) Hepatology 47:648-658). Nine days after cytokine gene delivery, replication-deficient adenovirus was intravenously injected into humice. Three days later, the liver was harvested and stained with H&E. Abundant leukocyte infiltration and large areas of necrosis were observed in the livers of IL-15- and FL-treated adenovirus-infected humice. However, nontreated humice infected with adenovirus exhibited only mild cell infiltration and damage (FIG. 4A). Correspondingly, the serum alanine aminotransferase (ALT) level was significantly elevated in IL-15- and FL-treated adenovirus-infected humice (FIG. 4B). This increase was correlated with an approximately fourfold increase in serum IFN-γ level (FIG. 4C) and an approximately fivefold increase in infiltrating human leukocytes in the livers (FIG. 4D). Immunohistochemical analysis of liver slices confirmed localization of CD56⁺ NK cells within the lesions (FIG. 4E). These results strongly suggest that cytokine-induced human NK cells are functional.

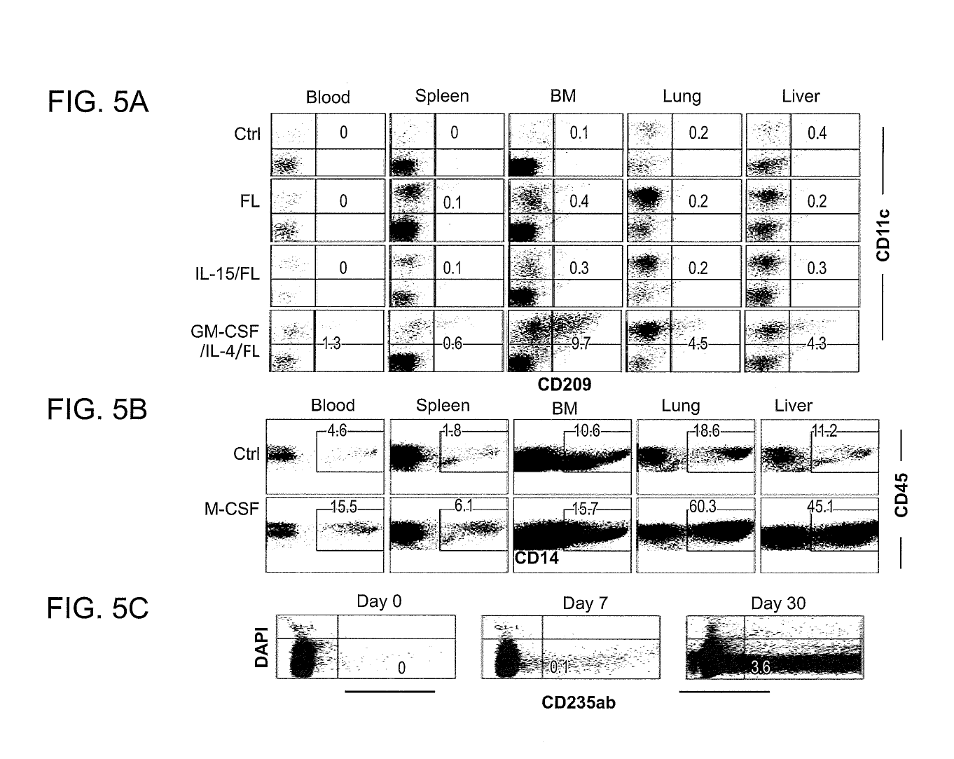

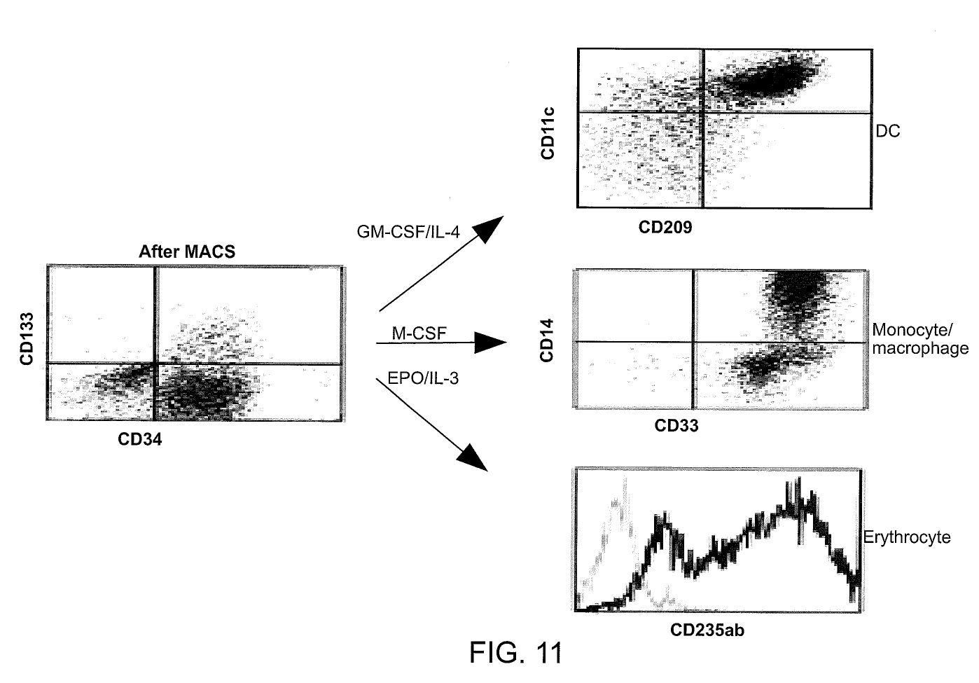

Improving Reconstitution of Other Human-Blood Cell Lineages. We tested whether cytokine gene delivery can be used as a general method to improve reconstitution of specific human-blood cell lineages in humice. In culture, human CD34⁺ cells purified from the BM of humice were stimulated to differentiate into CD11c⁺CD209⁺DCs by GM-CSF and IL-4, into CD14⁺ monocytes/macrophages by M-CSF, and into CD235ab⁺ erythrocytes by EPO and IL-3 (FIG. 11). In vivo, hydrodynamic delivery of DNA vectors expressing GM-CSF, IL-4, and FL into humice resulted in markedly increased frequency of CD11c⁺CD209⁺ DCs in the blood, spleen, BM, lung, and liver (FIG. 5A). Similarly, expression of M-CSF led to improved reconstitution of CD14+ monocytes/macrophages in both lymphoid and nonlymphoid organs (FIG. 5B). Expression of EPO and IL-3 resulted in the appearance of CD235ab⁺ human erythrocytes in the blood (FIG. 5C), reaching 3 to 5% of all red blood cells. Thus, cytokine gene expression by hydrodynamic injection of DNA plasmids is a general and efficient method to improve reconstitution of specific human-blood cell lineages in humice.

### Discussion

Reconstitution of NK cells and myeloid cells are generally poor in the humanized mouse models using NSG or BALB/c-Rag2^{-/-} Il2rg^{-/-} mice as recipients. In BLT mice, human NK cells and RBC are absent, despite significant reconstitution of DCs and monocyte/macrophage. We noticed that many cytokines, including IL-15, GM-CSF, 1L-4, M-CSF, and IL-3, required for NK cell or various myeloid cell development and maintenance, show significant sequence divergence between human and mouse. Previous studies have documented that these murine cytokines have little effect on appropriate human cell types. Because these cytokines are predominantly produced by nonhematopoietic cells, the lack of these human cytokines could explain the poor reconstitution of NK cells and myeloid cells in humice.

Supporting this interpretation, we showed that human CD34+ precursor cells isolated from the BM of humice can be stimulated in vitro to differentiate into NK cells, DCs, monocytes/macrophages, and erythrocytes. When appropriate human cytokines are introduced in the humanized mice by hydrodynamic delivery of cytokine-encoding plasmid DNA, significantly elevated levels of NK cells, DCs, monocytes/macrophages, and erythrocytes are induced. As the serum level of cytokines declines, the level of reconstitution also declines. Thus, the poor reconstitution of NK cells and myeloid cells in humice is a result of the lack of appropriate human cytokines required for their differentiation and maintenance. Introduction of appropriate cytokines leads to a dramatic increase in reconstitution levels of these human-blood cell lineages in humice.

Hydrodynamic gene delivery is widely used to produce high level, transient hepatic and systemic transgene expression in mice (Suda T, Liu D (2007) Mol Ther 15:2063-2069). The method involves tail-vein injection of DNA in a large volume (10% body weight) in a short duration (6-8 s). The hydrodynamic pressure causes liver damage, leading to uptake of DNA by hepatocytes (Suda T, Liu D (2007) Mol Ther 15:2063-2069). Following transcription and translation, cytokines are secreted into the circulation and can reach the target cells in the BM or other organs. Thus, with a single injection of cytokine encoding DNA, IL-15 was detected in the serum for 2 weeks and FL for 3 weeks. The difference between serum duration of IL-15 and FL is probably because of difference in the protein's half-life or that IL-15 is normally bound on the cell surface via IL-15Ra chain (Mortier E, Woo T, Advincula R, Gozalo S, Ma A (2008) J Exp Med 205:1213-1225). The amount of IL-15 and FL produced from a single DNA injection is apparently sufficient to induce a markedly elevated level of NK cells for at least 30 days. The persistence of NK cells when the cytokines were no longer detected in the circulation indicates that the critical role of the cytokines is exerted at an early stage of the differentiation. Once generated, NK cells are able to survive for an extended time after cytokines become undetectable in the circulation. Because of their effect on multiple blood-cell lineages, expression of FL and IL-15 also lead to elevated levels of monocytes/macrophages, DCs, and B cells, but not T cells, in the spleen and BM. Furthermore, expression of appropriate cytokines by hydrodynamic gene delivery also markedly enhances the reconstitution of specific myeloid lineage cells, including DCs, monocytes/macrophages, and erythrocytes, demonstrating the broad utility of the approach. Compared to the improved reconstitution of NK cells, DCs and monocytes/macrophages, which became apparent 7 days after delivery of human cytokine genes, improved reconstitution of erythrocytes did not reach the peak level until 30 days after cytokine gene delivery. This can be explained by the difference in the ratios of human WBC versus mouse WBC on the one hand, and human RBC versus mouse RBC on the other. Because of the large numbers of mouse RBC, it requires a longer time to produce sufficient numbers of human RBC to reach a similar percentage. Previously, two groups have reported enhanced NK cell development by injecting recombinant human IL-15 into NOD-scid mice or BALB/c-Rag2^{-/-} Il2rg^{-/-} mice (Huntington N D, et al. (2009) J Exp Med 206:25-34; Kalberer C P, Siegler U, Wodnar-Filipowicz A (2003) Blood 102:127-135). Compared to the daily cytokine injection, which is cumbersome and expensive, expression of cytokine genes by hydrodynamic gene delivery is an affordable, simple, and efficient method, as a single injection leads to elevated reconstitution of specific blood-cell lineages for more than 30 days.

The cytokine-induced NK cells exhibit normal surface phenotype and function. In contrast to a previous observation, where human NK cells were generated following daily injection of recombinant IL-15 in NOD-scid mice, the cells expressed NKp46 but not NKG2D and NKG2A (Kalberer CP, Siegler U, Wodnar-Filipowicz A (2003) Blood 102:127-135). In the present study, cytokine-induced human NK cells expressed all three major families of NK receptors, including activating receptor NKG2D, inhibitory receptors NKG2A and KIR, and the natural cytotoxicity receptor NKp46. Consistently, cytokine-induced NK cells are capable of lysing MHC class I-deficient target cells and secreting IFN-γ upon poly(I:C) stimulation both in vitro and in vivo. Furthermore, cytokine-induced NK cells are capable of mounting a robust response against adenovirus infection as indicated by the extensive liver necrosis and the high level of serum ALT in IL-15- and FL-treated humice. Similar to wild-type NK cells in mice, which mediate hepatitis by IFN-γ secretion (Chen Q, Wei H, Sun R, Zhang J, Tian Z (2008) Hepatology 47:648-658; Rosenberger C M, Clark A E, Treuting P M, Johnson C D, Aderem A (2008) Proc Natl Acad Sci USA 105:2544-2549), the levels of serum IFN-γ of IL-15- and FL-treated adenovirus-infected humice were significantly elevated. These findings suggest that cytokine-induced NK cells are normal in both surface phenotype and function.

### Example 2

### Expression of Human Cytokines Improves Reconstitution and Function of Human T and B Cells in Humanized Mice

The reconstitution of human T and B cells is reasonable in humanized mice, but they don't exhibit optimal functions. For example, although human CD8⁺ T cell response has been detected following viral challenges, the functions of CD4⁺ T cells are abnormal; human B cell mediated antibody response is absent in humanized mice. As shown herein, the abnormality of human T and B cell response is also due to the poor cross-reactivity between mouse cytokines and human cells in mice. Shown herein is that injection of human GM-CSF and IL-4 encoding plasmids into humice led to the improved reconstitution of human CD209⁺ dendritic cells, which is considered to be the major antigen presenting cells for T cells. Furthermore, IL-4 also was shown to promote cell proliferation, survival, and immunoglobulin class switch to IgG and IgE in human B cells, and acquisition of the Th2 phenotype by naïve CD4+ T cells. Toxoid (TT) vaccine was used to immunize the GM-CSF and IL-4 treated humice to determine whether these mice can generate TT specific antibody response.

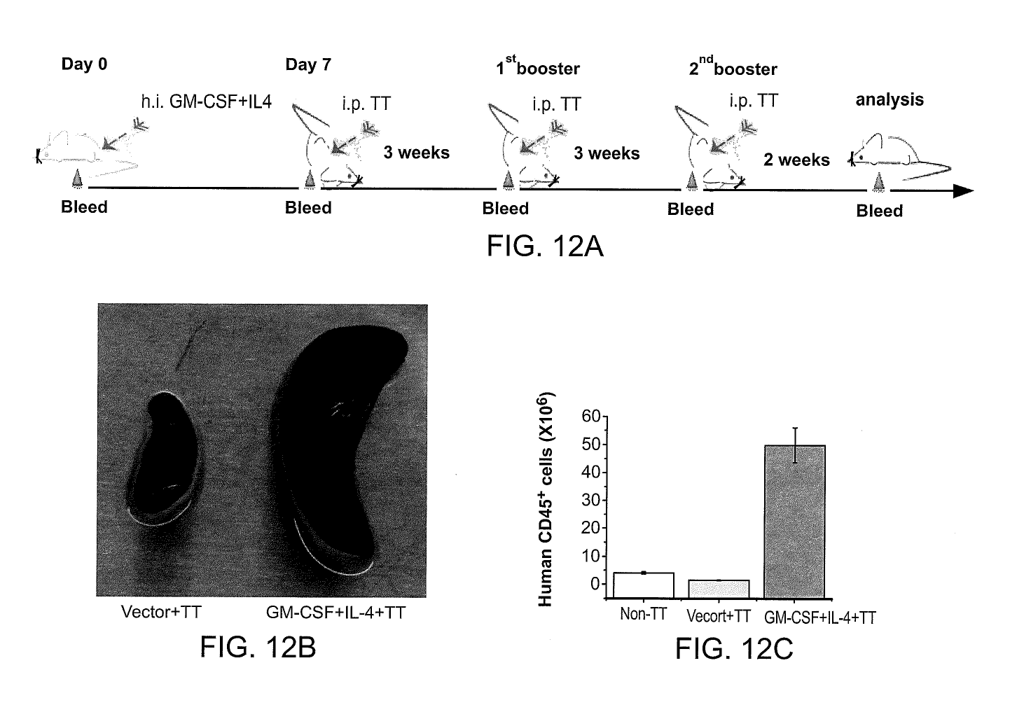

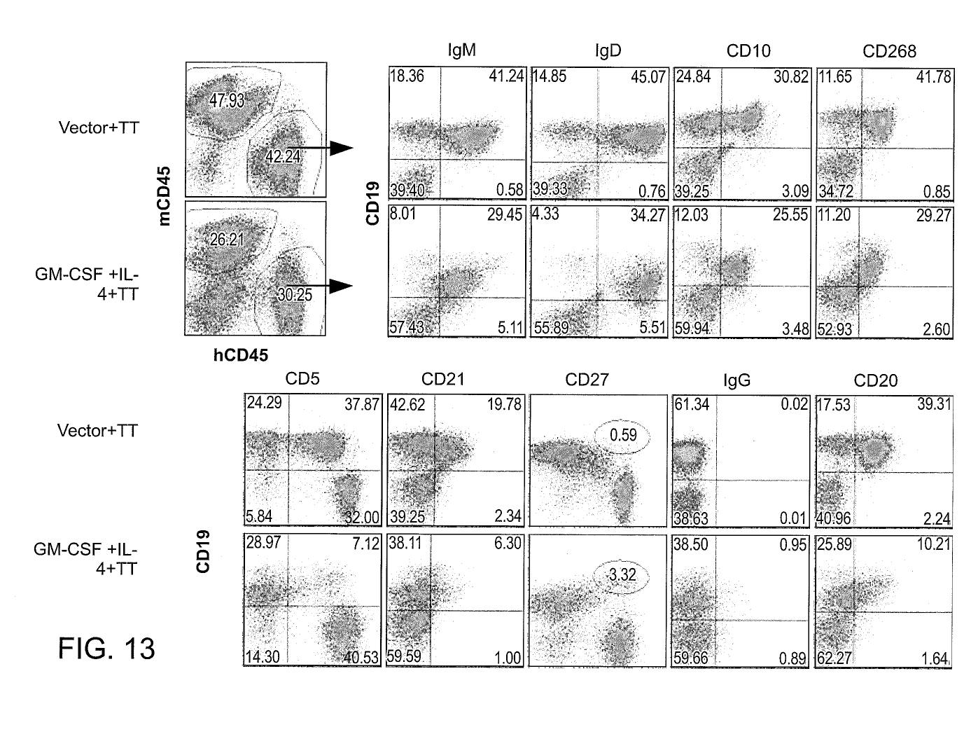

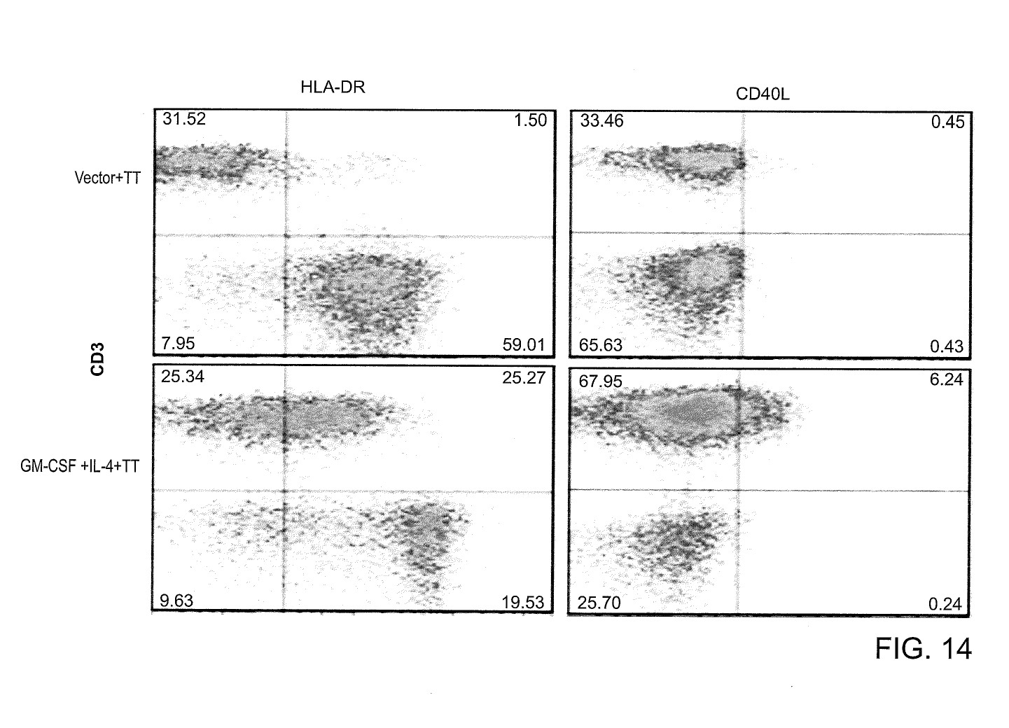

As described herein, 12-week-old humanized mice with similar human leukocyte reconstitution (50-80%) were hydrodynamically injected with plasmids encoding human GM-CSF and IL-4 or blank pcDNA vector (vector). After seven days, these mice were immunized with tetanus toxoid (TT) three times with 3 weeks intervals between doses (FIG. 12A). Spleens and sera were collected 2 weeks after the third immunization. FIG. 12B shows that the spleens from GM-CSF and IL-4 treated mice enlarged significantly compared to the vector treated mice. Correspondingly, there was a dramatic expansion of human mononuclear cells (MNCs) (around 20 fold) in the spleens of GM-CSF and IL-4 treated mice (FIG. 12C). From the cell surface phenotyping results of human B cells and T cells in spleens, it also indicated that the human B cells developed to a mature antibody-producing stage (CD19^{low}CD20⁻) which is identical to the normal human profile through the whole course of cytokine treatment and immunization (FIG. 13); meanwhile the human T cells were activated by up-regulating the expression of HLA-DR and CD40L (FIG. 14).

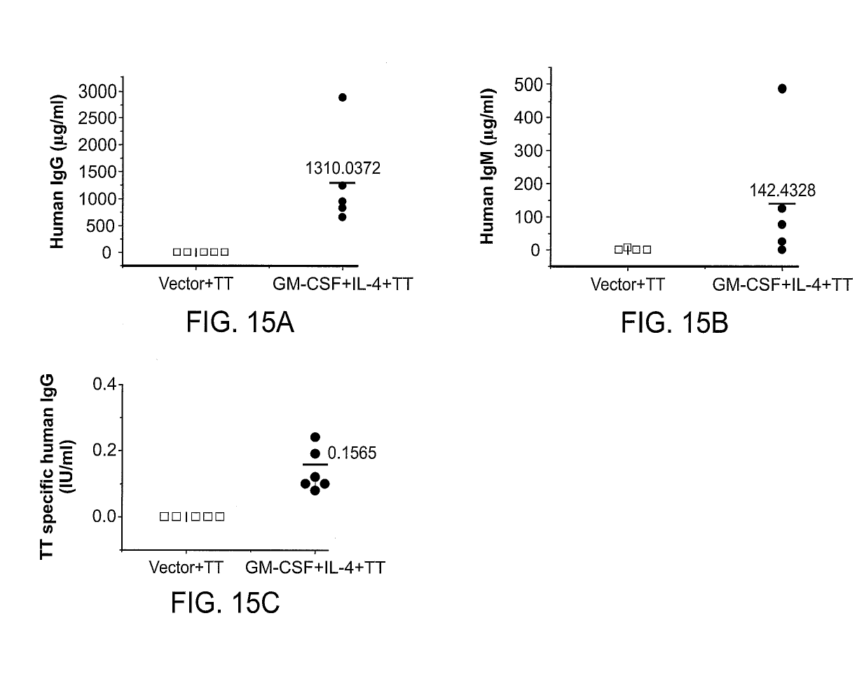

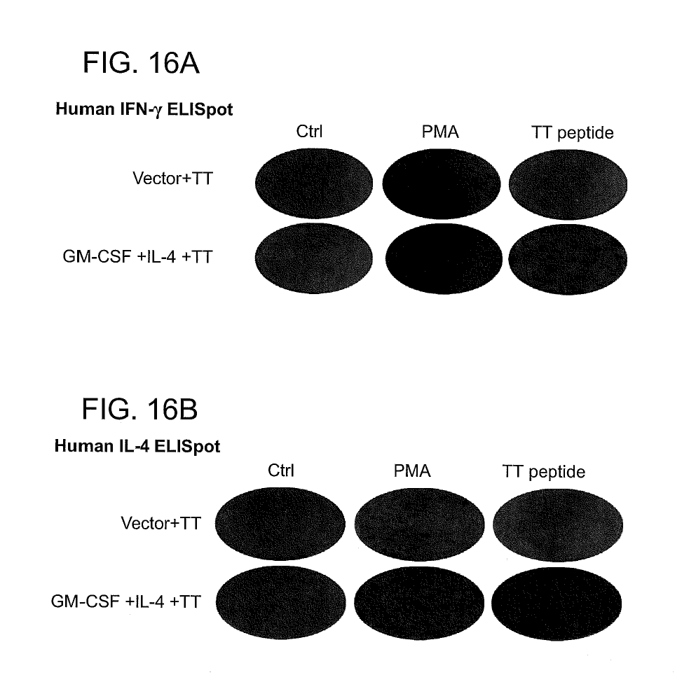

The total human IgG and IgM levels in the sera from the immunized, GM-CSF and IL-4 treated mice reached as high as 1.3 mg and 140 μg respectively (FIGS. 15A, 15B), which is similar to the levels in human (4 mg and 1 mg respectively). Most importantly, antigen specific human antibody responses were for the first time successfully established in humice. Human TT specific IgG was not detectable in vector treated mice while in the cytokines treated mice, it reached an average of 0.16 IU/ml (FIG. 15C). 0.1 IU/ml of anti-tetanus toxoid antibody in humans following immunization is sufficient to protect the individual from infection. Furthermore, the human T cell responses in GM⁻CSF⁻IL-4 treated mice also showed their specificity to TT antigen (FIGS. 16A-16B). A tetanus toxin peptide (830-843) was used to stimulate the spleen T cells. The T cells from GM⁻CSF⁺IL-4 treated mice were able to produce significant levels of human IFN-γ and IL-4 following the TT specific stimulation, compared to the cells from vector treated mice.

Using the methods described herein, significant levels of human antigen-specific antibody response can be established in mice. Thus, the methods described herein provide a useful platform for testing vaccines and producing human antibodies for therapeutic purposes.

TABLE
Cytokines and Cytokine Functions

| Cytokine | Function |
|---|---|
| IL-1alpha | Inflammation |
| IL-1beta | Inflammation |
| IL-2 | T cell/Treg |
| IL-3 | HSC |
| IL-4 | Th2, B cell, Dendritic cell |
| IL-5 | eosinophils |
| IL-6 | inflammation, hematopoiesis |
| IL-7 | Thymocyte, T cell |
| IL-8 | Neutrophils |
| IL-9 | T cell |
| IL-10 | Th2, autoimmune inflammation |
| IL-11 | HSC, B cell |
| IL-12 | Th1, NK |
| IL-13 | Macrophage, B cell |
| IL-14 | B cell proliferation |
| IL-15 | NK, B cell, T cell |
| IL-16 | CD4+ cells |
| IL-17 | Th17 |
| IL-18 | Th1 |
| IFN-g | B cell, macrophage, Th1 |
| IL-19 | Th2, monocyte |
| IL-20 | Keratinocytes, HSC |
| IL-21 | T, B, NK |
| IL-22 | inflammation |
| IL-23 | Th23, CD8⁺DC |
| IL-24 | Monocyte, dendritic cell |
| IL-25 | Th2 |
| IL-26 | T cell |
| IL-27 | T and B cell |
| IL-28 | Anti-viral response |
| IL-29 | Anti-viral and microbe |
| IL-30 | One chain of IL-27 |
| IL-31 | Th2, monocyte |
| IL-32 | Monocyte, macrophage |
| IL-33 | Th2 |
| IL-34 | Myeloid cells |
| IL-35 | Treg |
| Oncostatin M | Liver development, hematopoiesis |
| Leukemia inhibitory factor | Myeloid leukemia cells |
| Ciliary neurotrophic factor | nervous system |
| Cardiotrophin 1 | heart diseases |
| TNF-α | Inflammation |
| B-cell activating factor (BAFF) | B cell |
| Fas ligand | apoptosis |
| Lymphotoxin (TNF-β) | CD8⁺T cell |
| RANKL | Dendritic cells |
| TRAIL | Apoptosis |
| IFN-α | NK, macrophage |
| IFN-β | NK, macrophage |
| Stem cell factor | Stem cell |
| GM-CSF | HSC, monocyte |
| M-CSF | Monocyte, macrophage |
| G-CSF | Granulocyte, stem cells |
| Osteopontin | Immune cells, autoimmune disease |
| Chemokines | chemotaxis |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of reconstituting functional human blood cell lineages in a non-human mammal comprising
a) introducing human hematopoietic stem cells (HSCs) and nucleic acid encoding one or more human cytokines into an immunodeficient non-human mammal; and
b) maintaining the non-human mammal under conditions in which the nucleic acid is expressed and the human HSCs differentiate into functional human blood cell lineages in the non-human mammal, thereby reconstituting functional human blood cell lineages in the non-human mammal.

2. (canceled)

3. (canceled)

4. (canceled)

5. (canceled)

6. The method of claim 1 wherein the nucleic acid encoding the one or more human cytokines is introduced as plasmid DNA using hydrodynamic injection.

7. The method of claim 1 wherein the one or more human cytokines are selected from the group consisting of interleukin-12 (IL-12), interleukin-15 (IL-15), Flt3L (Fms-related tyrosine kinase 3 ligand), granulocyte macrophage colony stimulating factor (GM-CSF), interleukin-4 (IL-4), interleukin-3 (IL-3), macrophage colony stimulating factor (M-CSF), erythropoietin (EPO) and a combination thereof.

8. The method of claim 1 wherein the non-human mammal is a mouse.

9. (canceled)

10. The method of claim 1 wherein the functional human blood cell lineages that are reconstituted are functional human myeloid cells, function human lymphoid cells or combinations thereof.

11. (canceled)

12. (canceled)

13. (canceled)

14. (canceled)

15. (canceled)

16. The method of claim 1 wherein the functional human blood cell lineages are functional human NK cells:
and the nucleic acid encodes human IL-15 and human Flt-3/Flk-2 ligand.

17. (canceled)

18. The method of claim 16 wherein about 5% to about 21% of luekocytes in peripheral blood of the non-human mammal are human NK cells.

19. The method of claim 18 wherein the expression of human NK cells is maintained for about 30 days in the non-human mammal.

20. The method of claim 1 wherein the functional human blood cell lineages are functional human dendritic cells and the nucleic acid encodes human GM-CSF and human IL-4.

21. (canceled)

22. The method of claim 20 further comprising introducing nucleic acid encoding human Flt-3/Flk-2 ligand.

23. The method of claim 1 wherein the functional human blood cell lineages are functional human moncytes/macrophages and the nucleic acid encodes human macrophage colony stimulating factor.

24. (canceled)

25. The method of claim 1 wherein the functional human blood cell lineages are functional human erythrocytes and the nucleic acid encodes human erythropoietin and human IL-3.

26. (canceled)

27. The method of claim 25 wherein the erythrocytes comprise about 3% to about 5% of all red blood cells in the non-human mammal.

28. The method of claim 1 wherein the functional human blood cell lineages are functional human T cells and human B cells and the one or more human cytokines are granulocyte macrophage colony stimulating factor (GM-CSF) and interleukin-4 (IL-4).

29. (canceled)

30. (canceled)

31. (canceled)

32. (canceled)

33. (canceled)

34. (canceled)

35. The method of claim 28 wherein the non-human mammal is a mouse.

36. (canceled)

37. The method of claim 28 further comprising
c) immunizing the non-human mammal with an immunogen; and
d) maintaining the non-human animal under conditions in which the non-human mammal produces human antibodies directed against the immunogen.

38. The method of claim 37 wherein the human antibodies are human IgG, human IgM or a combination thereof.

39. The method of claim 38 further comprising isolating human B cells that produce the human antibodies from the non-human mammal, thereby producing isolated human B cells.

40. The method of claim 39 further comprising contacting the isolated human B cells with immortalized cells, thereby producing a combination; and maintaining the combination under conditions in which the human B cells and the immortalized cells fuse to form a hybridoma that produces monoclonal antibodies directed against the immunogen.

41. (canceled)

42. A method of generating human antibodies directed against an immunogen in a non-human mammal comprising
a) introducing into an immunodeficient non-human mammal human hematopoietic stem cells (HSCs) and nucleic acid encoding one or more human cytokines into the non-human mammal wherein the human cytokines promote differentiation of the human HSCs into functional human T cell and human B cells;
b) maintaining the non-human mammal under conditions in which the nucleic acid is expressed and the HSCs differentiate into functional human T cells and human B cells in the non-human mammal;
c) immunizing the non-human mammal with the immunogen; and
d) maintaining the non-human animal under conditions in which the human B cells produce human antibodies directed against the immunogen in the non-human mammal, thereby generating human antibodies directed against the immunogen in the non-human mammal.

43. (canceled)

44. (canceled)

45. (canceled)

46. (canceled)

47. The method of claim 42 wherein the nucleic acid encoding the one or more human cytokines is introduced as plasmid DNA using hydrodynamic injection.

48. The method of claim 42 wherein the one or more human cytokines are granulocyte macrophage colony stimulating factor (GM-CSF) and interleukin-4 (IL-4).

49. The method of claim 42 wherein the non-human mammal is a mouse.

50. (canceled)

51. (canceled)

52. The method of claim 42 further comprising isolating human B cells that produce the non-human the human antibodies from the non-human mammal, thereby producing isolated human B cells.

53. The method of claim 52 further comprising contacting the isolated human B cells with immortalized cells, thereby producing a combination; and maintaining the combination under conditions in which the human B cells and the immortalized cells fuse to form a hybridoma that produces monoclonal antibodies directed against the immunogen.

54. (canceled)

55. (canceled)

56. A non-human mammal produced by the method of claim 1.

57. (canceled)

58. (canceled)

59. (canceled)

60. (canceled)

61. (canceled)

62. A hybridoma produced by the method of claim 40.

63. A monoclonal antibody secreted by the hybridoma of claim 62.

64. A hybridoma produced by the method of claim 53.

65. A monoclonal antibody secreted by the hybridoma of claim 64.
